(19) United States
(12) Patent Application Publication
(10) Pub. No.: US 2004/0029825 A1
(43) Pub. Date: Feb. 12, 2004

(54) Methods of minimizing immunological rejection of a nuclear transfer fetus

(76) Inventors: Christopher J Davies, Pullman, WA (US); Donald H Schlafer, Alpine, NY (US); Jonathan R HIll, New South Wales (AU)

Correspondence Address: Michael L Goldman, Nixon Peabody, P O Box 31051, Clinton Square, Rochester, NY, 14603, US

(21) Appl. No.: 10/398,308

(22) PCT Filed: Oct. 3, 2001
(86) PCT No.: PCT/US01/30925

(51) Int. Cl. A61K 48/00

(52) U.S. Cl. 514/44; 800/21

(57) ABSTRACT

The present invention relates to a method of minimizing immunological rejection of a nuclear transfer (“NT”) fetus which includes transferring a NT embryo into an embryo recipient under conditions effective for development of a NT fetus with minimal risk of immunological rejection of the fetus due to a maternal anti-fetal MHC-I immune response. After determining an MHC-I antigen type for a NT embryo and an MHC-I antigen type for embryo recipients, the NT embryo is either (i) transferred into a first embryo recipient having a compatible MHC-I antigen type under conditions effective for development of a NT fetus from the NT embryo, or (ii) transferred into a second embryo recipient having an incompatible MHC-I antigen type, followed by regulating MHC-I expression of the NT embryo or suppressing an immune response of the embryo recipient under conditions effective for development of a nuclear transfer fetus.

This application claims the benefit of U.S. Provisional Application Serial No. 60/237,673, filed Oct. 3, 2000, which is hereby incorporated by reference in its entirety.

This invention was made, at least in part, with funding received from the U.S. Department of Agriculture, NRICGP, Grant No. 96-35203-3356. The U.S. government may have certain rights in this invention.

### FIELD OF THE INVENTION

The present invention relates generally to animal cloning and, more specifically, to methods of minimizing immunological rejection of a nuclear transfer (“NT”) fetus.

### BACKGROUND OF THE INVENTION

Success has now been achieved with somatic cell cloning in several species using a variety of cell types (Campbell et al., 1996; Schnieke et al., 1997; Wells et al., 1997; Wilmut et al., 1997; Cibelli et al., 1998; Kato et al., 1998; Wakayama et al., 1998; Baguisi et al., 1999; Renard et al., 1999; Wells et al., 1999; Wakayama, Yanagimachi, 1999a). This technology has great potential for use in agriculture, animal and human medicine, and for the propagation of rare animals. These potential uses of cloning technology clearly have great commercial and conservational benefit.

The efficiency of this process, however, is quite poor, resulting in less than one animal born per 100 reconstructed NT embryos (Schieke et al., 1997). Much of this inefficiency is due to low initial pregnancy rates and early pregnancy losses. First trimester losses of greater than 50% are common for nuclear transfer pregnancies (Wells et al., 1997; Wilmut et al., 1997; Cibelli et al., 1998; Baguisi et al., 1999; Wells et al., 1999), whereas only 2-4% of naturally conceived Day 30 bovine pregnancies and 11% of in vitro produced embryos are expected to be lost by Day 60 (Alexander et al., 1995; Hasler et al., 1995; Forar et al., 1996). This lack of normal in vivo development has occurred in each species so far studied and is delaying the transfer of this new technology into commercial practice.

In general, early fetal losses may be due to abnormalities of the embryo or its placenta, alterations in maternal uterine environment or feto-maternal interactions (Wilmut et al., 1986). In normal pregnancies, fetal abnormalities are known to be a major cause of pregnancy loss (Wilmut et al., 1986). Fetal abnormalities, predominantly fetal oversize, have been observed as a result of in vitro embryo culture and this syndrome is believed to result from serum containing media (Thompson et al., 1995; Walker et al., 1996; Young et al., 1998). In sharp contrast, NT fetuses that die during the first trimester are undersized, which probably represents the effects of “starvation” due to inadequate maternal-fetal contact and poor transfer of nutrients (Hill et al. 2000b). The fetuses that die appear not to lose viability because of inherent fetal problems, but due to starvation from an inadequate placental nutrient transfer.

In cloned animals, normal placental development appears to be rare, as placental abnormalities occur at a high incidence in early and late term cloned fetuses (Stice et al., 1996; Hill et al., 1998; Wells et al., 1998). Stice et al. (1996) observed a lack of placentome development in Day 35-50 cloned bovine fetuses and suggested that this caused a high rate of first trimester death. Even in NT fetuses that survive beyond Day 50, the number of placentomes may be reduced from normal by as much as 80% (Hill et al., 1998). This suggests that the completeness of placental development in cloned animals varies widely.

King et al. (1979) documented the normal development of Day 30-60 bovine placentas. The placental attachment phase in ruminants is progressive and extends almost throughout the first trimester in contrast to the more rapid and invasive attachment phase in humans and rodents. At Day 30, placentomes are visible using light microscopy with tenuous attachment of maternal and fetal epithelia and formation of microvilli. Contact with the maternal caruncle areas of the endometrium induces growth of villous processes that undergo hypertrophy and hyperplasia to form cotyledons (Noden, de Lahunta, 1990) and by Day 42 larger, more complex placentomes develop (King et al., 1979). Placentomes are formed from extensive and complex branching of fetal villi and maternal crypts, serve as specialized areas for supplying nutrition to the developing conceptus. Villous projections assist in maintaining apposition and facilitate subsequent union. Binucleate cells form transient feto-maternal syncytia in the cow, which has been proposed to be central to villous expansion (Wooding, Flint, 1994). Chorioallantoic villous formation at the cotyledons is thought to be the primary site of transport of easily diffusible small molecules such as oxygen, carbon dioxide and also amino acids and glucose, whereas macromolecules are transported in the interplacentomal areas adjacent to uterine gland openings.

Cloned pregnancies fail at a higher than normal rate during each trimester of pregnancy (Wilmut et al. 1997). Although in vitro development rates to the blastocyst stage approach that of in vitro fertilized (“IVF”) embryos, subsequent in vivo development drops dramatically. Pregnancy rates at Day 30 in recipient cows can approach 50%, but only with transfer of two or more cloned embryos. Single embryo transfer of cloned embryos results in almost negligible pregnancy rates whereas IVF embryos transferred singly achieve 50-70% pregnancies. The cause of these losses have not been determined and may be due to failure of maternal recognition, placental development, or inherently low cloned embryo viability.

Following on from these losses is a well documented period of embryonic loss from Day 30-60 that results in a minority of first trimester pregnancies maintaining their viability into the second trimester (Wells et al., 1997; Wilmut et al., 1997; Cibelli et al., 1998; Baguisi et al., 1999; Wells et al., 1999) Hill et al. 2000b). During the second and third trimesters there are sporadic losses of cloned fetuses, often accompanied by the development of major placental abnormalities such as hydrops allantois. Postnatal viability is also markedly lower for many cloned calves (Kato et al. 1998; Hill et al. 1999a; Renard et al. 1999; Kubota et al. 2000; Kato et al. 2000). It is uncertain if the cause(s) of fetal loss in the first trimester is related to later losses.

For somatic cell NT to become a viable technique, its efficiency must be improved. Although the numbers of cloned calves born worldwide since 1998 has rapidly increased into the hundreds and press reports often detail the latest successful birth, these successes gloss over the huge amount of resources that must be devoted to producing each cloned calf. If the cloning technique can be improved so that pregnancy rates increase and fetal losses decrease to approximate those of in vitro produced embryos and fetuses, noted above, utilization of the technique would immediately increase. This would enable the use of cloning in commercial agriculture, facilitate production of transgenic animals, and dramatically reduce the costs to research institutions in maintaining recipient cows for cloned embryos.

The present invention is directed to overcoming the above-noted deficiencies in art and otherwise minimizing the failure of NT pregnancies.

### SUMMARY OF THE INVENTION

A first aspect of the present invention relates to a method of minimizing immunological rejection of a nuclear transfer fetus which includes transferring a nuclear transfer embryo into an embryo recipient under conditions effective for development of a nuclear transfer fetus with minimal risk of immunological rejection of the fetus due to a maternal anti-fetal MHC class I (“MHC-I”) immune response.

A second aspect of the present invention relates to a method of performing embryo transfer which includes: determining an MHC-I antigen type for a nuclear transfer embryo and an MHC-I antigen type for embryo recipients and either (i) transferring the nuclear transfer embryo into a first embryo recipient having a compatible MHC-I antigen type under conditions effective for development of a nuclear transfer fetus from the nuclear transfer embryo, or (ii) transferring the nuclear transfer embryo into a second embryo recipient having an incompatible MHC-I antigen type and (a) regulating MHC-I expression of the nuclear transfer embryo or (b) suppressing an immune response of the embryo recipient, under conditions effective for development of a nuclear transfer fetus from the nuclear transfer embryo. Ultimately, development of a healthy neonate from the nuclear transfer fetus/embryo is desired.

A third aspect of the present invention relates to an MHC-I microarray typing system which includes: a substrate and a plurality of oligonucleotide probes bound to the substrate, each of the plurality of oligonucleotides binding to at least one MHC-I allele, wherein each MHC-I allele binds to different oligonucleotide probes.

Trophoblast cells in NT embryos display abnormal expression of MHC-I antigen. The abnormal MHC class I expression results in immunological rejection of these fetuses in a large proportion of NT pregnancies, particularly during the first trimester. This is in sharp contrast to normal pregnancies, where the rate of early embryonic loss is low and MHC incompatible pregnancies do not have a significantly increased amount of early embryonic loss. Presumably, the reason for this distinction is that in normal bovine pregnancy, there is no trophoblast MHC-I antigen expression in early pregnancy (Davies et al., 2000). Consequently, MHC-I antigen expression is not a target for immunologically mediated fetal rejection in normal pregnancies. To overcome this problem acutely associated with NT transfer, the present invention identifies two approaches for avoiding immunological rejection of MHC-I incompatible NT pregnancies. The first approach involves matching NT donor cells and NT recipients for their MHC-I haplotype expression prior to transfer. According to a second approach, which can be employed independently of the first approach (i.e., with or without prior matching), MHC-I antigen expression by NT trophoblasts is down-regulated, returning the NT trophoblasts to their normal MHC-I negative state. Both of these approaches minimize rejection of the NT fetus, particularly during the first trimester.

### BRIEF DESCRIPTION OF THE DRAWINGS

FIGS. 1A-J illustrate the nucleotide sequence alignment of different MHC-I alleles. This sequence alignment was prepared by George Russell of the Roslin Institute and made available at the Internet site for the Bovine Leucocyte Antigens (BOLA) nomenclature committee (standing committee of the International Society for Animal Genetics). d18-2 (SEQ ID No: 1, Genbank Accession No. Y09206); a10 (SEQ ID No: 2, Genbank Accession No. M69026); jsp1 (SEQ ID No: 3, Genbank Accession No. X92870); pbola1 (SEQ ID No: 4, Genbank Accession No. M24090); bl3-6 (SEQ ID No: 5, Genbank Accession No. M21044); bsa (SEQ ID No: 6, Genbank Accession No. L02832); bsc (SEQ ID No: 7, Genbank Accession No. L02833); d18-1 (SEQ ID No: 8, Genbank Accession No. Y09205); bsn (SEQ ID No: 9, Genbank Accession No. L02835); man1 (SEQ ID No: 10, Genbank Accession No. AJ010863); bsf (SEQ ID No: 11, Genbank Accession No. L02834); man8 (SEQ ID No: 12, Genbank Accession No. AJ010866); d18-3 (SEQ ID No: 13, Genbank Accession No. Y09207); pbolal19 (SEQ ID No: 14, Genbank Accession Nos. X82671-X82675); bl3-7 (SEQ ID No: 15, Genbank Accession No. M21043); hd7 (SEQ ID No: 16, Genbank Accession No. X80935); hd6 (SEQ ID No: 17, Genbank Accession No. X80934); 3349 (SEQ ID No: 18, Genbank Accession No. AJ010862); man2 (SEQ ID No: 19, Genbank Accession No. AJ010861); man3 (SEQ ID No: 20, Genbank Accession No. AJ010864); 4221 (SEQ ID No: 21, Genbank Accession No. AJ010865); hd1 (SEQ ID No: 22, Genbank Accession No. X80933); bsx (SEQ ID No: 23, Genbank Accession No. U01187); d18-4 (SEQ ID No: 24, Genbank Accession No. Y09208); pbola4 (SEQ ID No: 25, Genbank Accession Nos. X87645 and X97646-X97649); kn104 (SEQ ID No: 26, Genbank Accession No. M69204); and hd15 (SEQ ID No: 27, Genbank Accession No. X80936). All Genbank Accessions are hereby incorporated by reference in their entirety.

FIGS. 2A-D are photomicrographs comparing normal and cloned embryo development. Photomicrographs were originally photographed at 200×. In FIG. 2A, the endometrium and attached chorioallantois from a normal bovine pregnancy are shown at 39 days gestation (H+E stain). Note trophoblast cells forming a pseudocolumnar layer of cells and the subjacent endometrium lined by an irregular layer of endometrial epithelial cells. Two endometrial glands and moderately cellular endometrial interstitium are evident in the endometrium. In FIG. 2B, the endometrium of a cow pregnant 35 days with a cloned embryo (fetal membranes are not shown; H+E stain) is shown containing a marked lymphoplasmacytic cellular infiltrate extending from just beneath the endometrial epithelium to deep within the endometrium. This is in marked contrast to the normal cellularity demonstrated in FIG. 2A. FIGS. 2C-D illustrate sections of normal day 39 chorioallantois and endometrium (2C) and day 35 cloned embryonic placenta and opposing maternal endometrium (2D), respectively. Immunohistochemistry staining was performed with ILA19 antibody for bovine MHC-I antigen. Note the mild staining of the endometrial epithelial cells and complete absence of staining of trophoblast cells in FIG. 2C. Contrast this to the intense class I staining of the trophoblast and endometrial cells in fetal and maternal tissues from a cow carrying a cloned fetus shown in FIG. 2D. The trophoblast and endometrial cells show marked upregulation of MHC-I expression.

FIG. 3 is a graph illustrating the interaggregate cd3 positive cells located in the endometrium of 3 cloned pregnancies (hatched bars) and 7 controls (clear bars). The counts are the number of cd3 positive cells per 0.584 mm² field at 10× magnification.

### DETAILED DESCRIPTION OF THE INVENTION

The present invention is directed to new approaches for performing nuclear transfer (“NT”) embryo transfer into embryo recipients and, as a result, minimizing the immunological rejection of a developing NT fetus. By minimizing the immunological rejection, it is intended that the incidence of NT fetus rejection when practicing an embodiment of the present invention is less than the historical incidence of NT fetus rejection, which is greater than about 80 percent for bovine during the first trimester (Hill et al., 2000a).

The NT embryo is prepared using donor and recipient cells from a non-human mammal, preferably a ruminant such as a cow, sheep, goat, buffalo, water buffalo, llama, alpaca, camel, giraffe, etc., or other mammals such as pig, horse, rabbit, mouse, or rat. Procedures for preparing the NT embryo are known in the art (Campbell et al., 1996; Schnieke et al., 1997; Wells et al., 1997; Wilmut et al., 1997; Cibelli et al., 1998; Kato et al., 1998; Wakayama et al., 1998; Baguisi et al., 1999; Renard et al., 1999; Wells et al., 1999; Wakayama, Yanagimachi, 1999a) and further been have been described in U.S. Pat. No. 6,147,276 to Campbell et al. and U.S. Pat. No. 6,235,970 to Stice et al.

Suitable donor cells, i.e., cells useful in the subject invention, may be obtained from any cell or organ of the body. This includes all somatic or germ cells. All cells of normal karyotype, including embryonic, fetal and adult somatic cells, may prove totipotent. Donor cells may be, but do not have to be, in culture. Cultured bovine primary fibroblasts, an embryo-derived ovine cell line (TNT4), an ovine mammary epithelial cell derived cell line (OME) from a 6 year old adult sheep, a fibroblast cell line derived from fetal ovine tissue (BLWF1), and an epithelial-like cell line derived from a 9-day old sheep embryo (SECL) have been employed for nuclear transfer and described elsewhere. A class of embryo-derived cell lines useful in the invention, which includes the TNT4 cell line, are also the subject of PCT Publication No. WO 96/07732 to Campbell et al. All can be utilized in the present invention.

Donor cells may be, but do not have to be quiescent. Cultured cells can be induced to enter the quiescent state by various methods including chemical treatments, nutrient deprivation, growth inhibition or manipulation of gene expression. Presently, the reduction of serum levels in the culture medium has been used successfully to induce quiescence in both ovine and bovine cell lines. In this situation, the cells exit the growth cycle during the G1 phase and arrest in the so-called G0 stage. Such cells can remain in this state for several days (possibly longer depending upon the cell) until re-stimulated when they re-enter the growth cycle. Quiescent cells arrested in the G0 state are diploid. The G0 state is the point in the cell cycle from which cells are able to differentiate.

The recipient cell to which the nucleus from the donor cell is transferred may be an oocyte or another suitable cell. Recipient cells at a variety of different stages of development can be used, from oocytes at metaphase I through metaphase II to zygotes and two-cell embryos. Methods for isolation of oocytes are well known in the art. Essentially, this includes isolating oocytes from the ovaries or reproductive tract of a mammal. A readily available source of bovine oocytes is slaughterhouse materials.

Typically, oocytes should be matured in vitro before these cells may be used as recipient cells for nuclear transfer. This process generally requires collecting immature (prophase 1) oocytes from mammalian ovaries and maturing the oocytes in a maturation medium prior to enucleation until the oocyte attains the metaphase II stage, which in the case of bovine oocytes generally occurs about 18-24 hours post-aspiration (the “maturation period”).

Additionally, metaphase II stage oocytes, which have been matured in vivo have been successfully used in nuclear transfer techniques. Essentially, mature metaphase II oocytes are collected surgically from either non-superovulated or superovulated cows or heifers 35 to 48 hours past the onset of estrus or past the injection of human chorionic gonadotropin (hCG) or similar hormone.

The stage of maturation of the oocyte at enucleation and nuclear transfer has been reported to be significant to the success of NT methods (Prather et al. 1991). In general, successful mammalian embryo cloning practices use the metaphase II stage oocyte as the recipient oocyte, because at this stage it is believed that the oocyte can be or is sufficiently “activated” to treat the introduced nucleus as it does a fertilizing sperm. In domestic animals, and especially cattle, the oocyte activation period generally ranges from about 10 to about 52 hours, preferably about 16 to about 42 hours post-aspiration.

Enucleation can be effected by known methods, such as described in U.S. Pat. No. 4,994,384 to Prather et al. For example, enucleation may be accomplished microsurgically using a micropipette to remove the polar body and the adjacent cytoplasm. The oocytes may then be screened to identify those of which have been successfully enucleated. This screening may be effected by staining the oocytes with 1 μg/ml 33342 Hoechst dye in HECM, and then viewing the oocytes under ultraviolet irradiation for less than 10 seconds. The oocytes that have been successfully enucleated can then be placed in a suitable culture medium, e.g., CR1aa plus 10% serum.

Once suitable donor and recipient cells have been identified, it is necessary for the nucleus of the former to be transferred to the latter. Suitable procedures for nuclear transfer include donor/recipient cell fusion (i.e., via PEG treatment, inactivated Sendai virus, or electrofusion) and microinjection.

In donor/recipient cell fusion protocols, the donor cell is first transferred into the perivitelline space of the enucleated oocyte. Thereafter, the cells can be fused by providing a pulse of electricity that is sufficient to cause a transient breakdown and subsequent reformation of the plasma membrane. If upon reformation the lipid bilayers intermingle, small channels will open between the two cells and, due to the thermodynamic instability of such a small opening, it enlarges until the two cells become one (U.S. Pat. No. 4,997,384 to Prather et al.). A variety of electrofusion media can be used including e.g., sucrose, mannitol, sorbitol and phosphate buffered solution. Alternatively, fusion can also be accomplished using Sendai virus as a fasogenic agent (Graham 1969).

In microinjection protocols, the donor nuclei is simply removed from the donor cell and injected into the recipient cell (Collas & Barnes 1994).

Either before or, preferably, after nuclear transfer (or in some instances concomitantly therewith), parthenogenetic activation is typically required, at least if the cell is an oocyte, to stimulate the recipient cell into development. Parthenogenic activation is typically achieved using electrical stimulation of the diploidized oocyte, which is believed to allow for increases in intracellular calcium concentration. There is evidence that the pattern of calcium transients varies with species and it can be anticipated that the optimal pattern of electrical pulses will vary in a similar manner. The interval between pulses for rabbit oocytes is approximately 4 minutes (Ozil 1990), and in the mouse 10 to 20 minutes (Cuthbertson & Cobbold 1985), while observations in the cow suggest that the interval is approximately 20 to 30 minutes (Robt et al. 1992). In most published experiments activation was induced with a single electrical pulse, but new observations suggest that the proportion of reconstituted embryos that develop is increased by exposure to several pulses (Collas and Robl 1990). In any individual case, routine adjustments may be made to optimize the number of pulses, the field strength and duration of the pulses, and the calcium concentration of the medium.

Alternative approaches for parthenogenic activation include culturing the recipient oocyte of NT embryo at sub-physiological temperature (e.g., room temperature) and chemical shock. Suitable oocyte activation methods are further described in U.S. Pat. No. 5,496,720 to Susko-Parrish et al.

By way of example, activation can be effected by briefly exposing the fused NT embryo to a TL-HEPES medium containing 5 μM ionomycin and 1 mg/ml BSA, followed by washing in TL-HEPES containing 30 mg/ml BSA within about 24 hours after fusion, and preferably about 4 to 9 hours after fusion.

The reconstituted NT embryo may then give rise to one or more mammals, whether transgenic or non-transgenic. Preferably, the NT embryo will be cultured to a size of at least 2 to 400 cells, preferably 4 to 128 cells, and most preferably to a size of at least about 50 cells. Development to blastocyst stage can be carried out in vitro or in vivo (i.e., using a temporary pre-blastocyst recipient).

After preparing the NT embryo (and optionally developing the NT embryo to the blastocyst stage), it is transferred into the uterus of an embryo recipient using known transfer procedures. The embryo recipient is preferably from the same species as the donor and recipient cells used to prepare the NT embryo, although dams from related species can, at least in some instances, be utilized to support gestation of the NT fetus. Synchronous transfers are desirable for success of the transfer, i.e., the stage of the NT embryo is in synchrony with the estrus cycle of the recipient female (Siedel 1981). Transfer procedures are described in detail in PCT Publication No. WO 94/26884 to Wheeler et al., PCT Publication No. WO 94/24274 to Smith et al., PCT Publication No. WO 90/03432 to Evans et al., U.S. Pat. No. 4,944,384 to Prather et al., and U.S. Pat. No. 5,057,420 to Massey.

According to one embodiment, the method for minimizing immunological rejection of a NT fetus involves transferring, into an embryo recipient, an NT embryo having an MHC-I antigen type which is compatible with an MHC-I antigen type of the embryo recipient. Prior to transferring the NT embryo, the MHC-I antigen type of the NT embryo and the embryo recipient are determined. Matching of the NT embryo and the embryo recipient (into which transfer will subsequently occur) is based on the determined MHC-I antigen haplotypes thereof.

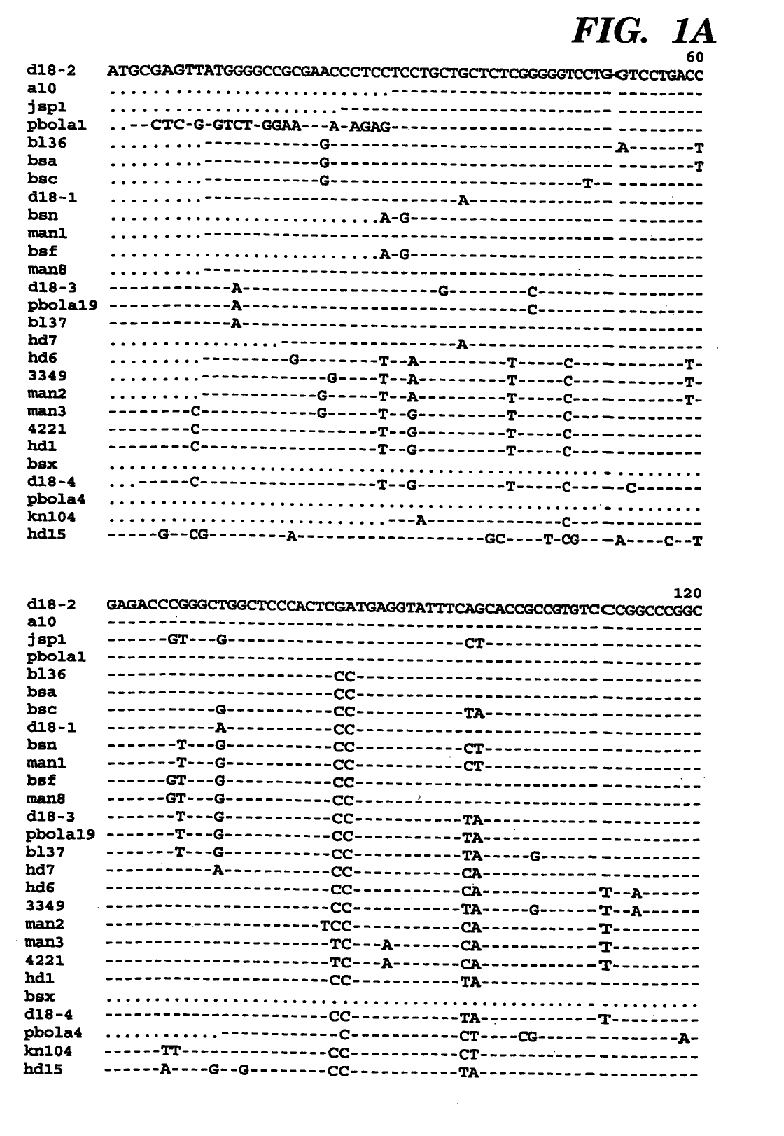

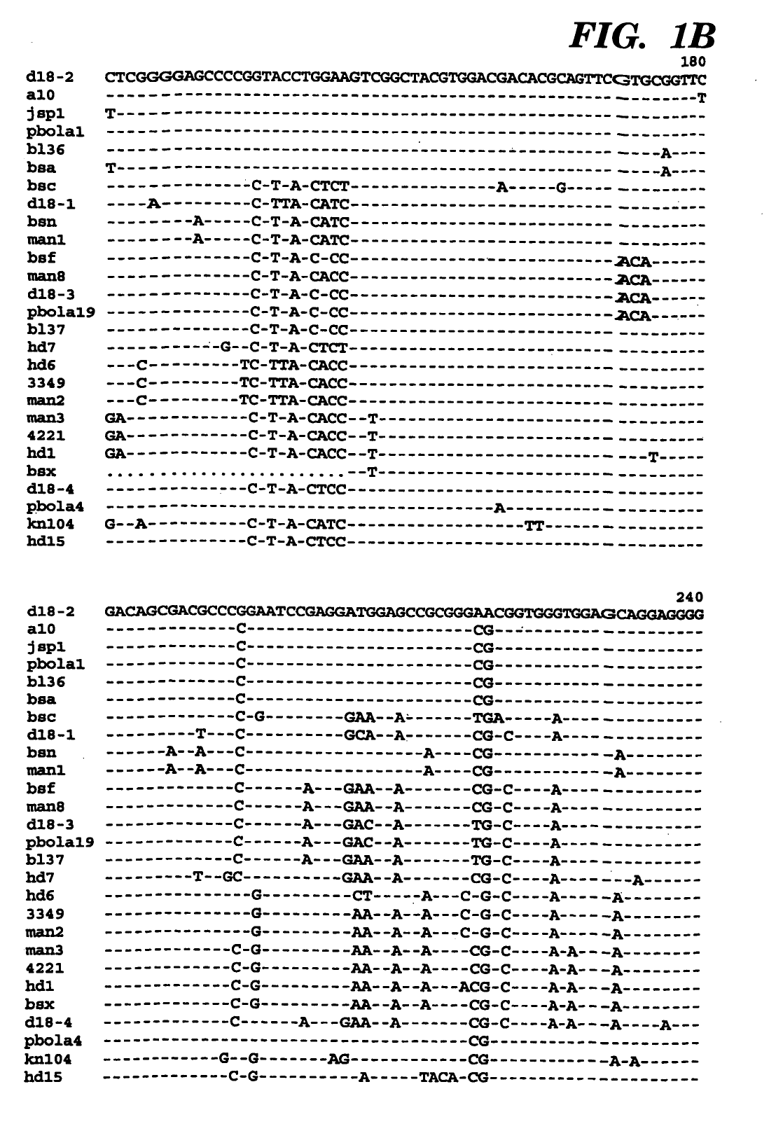

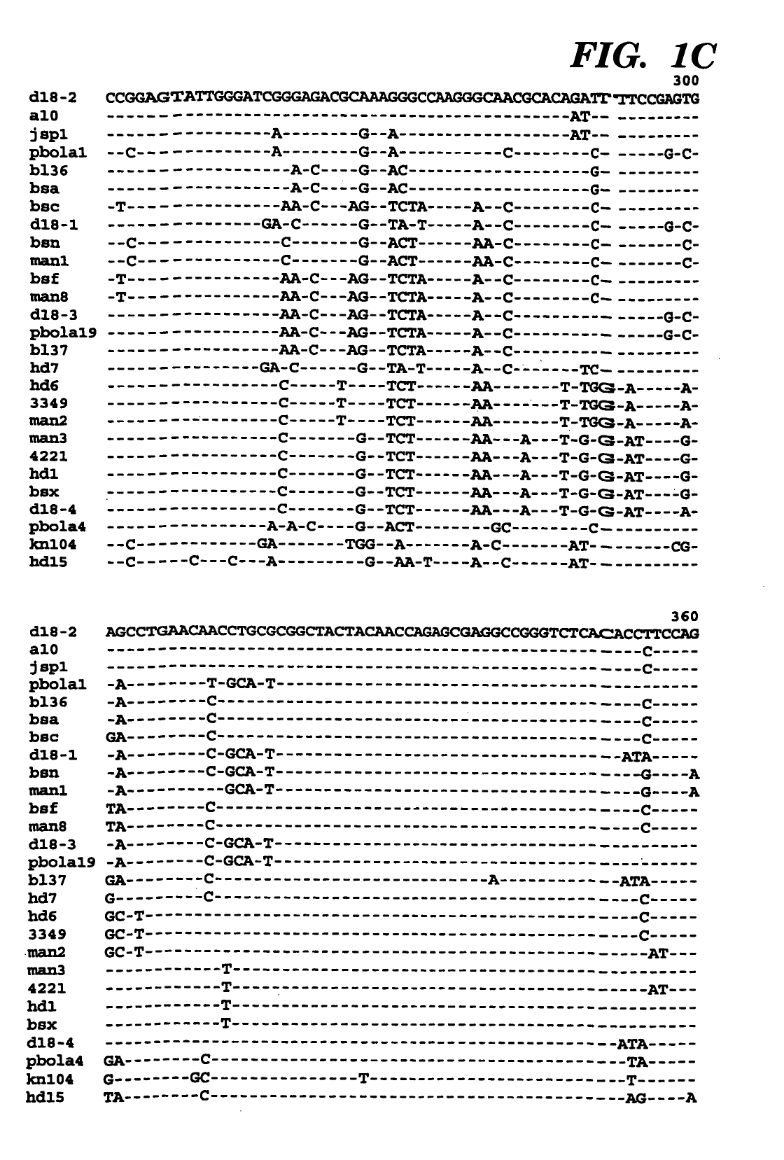

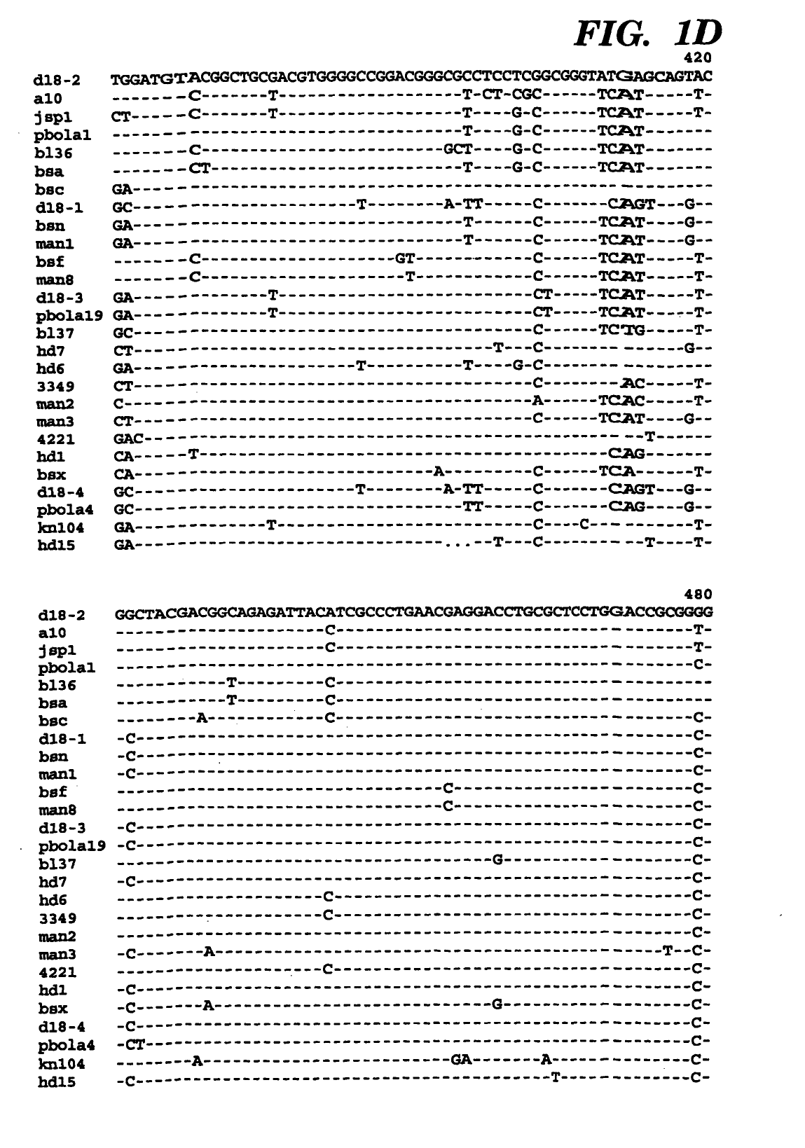

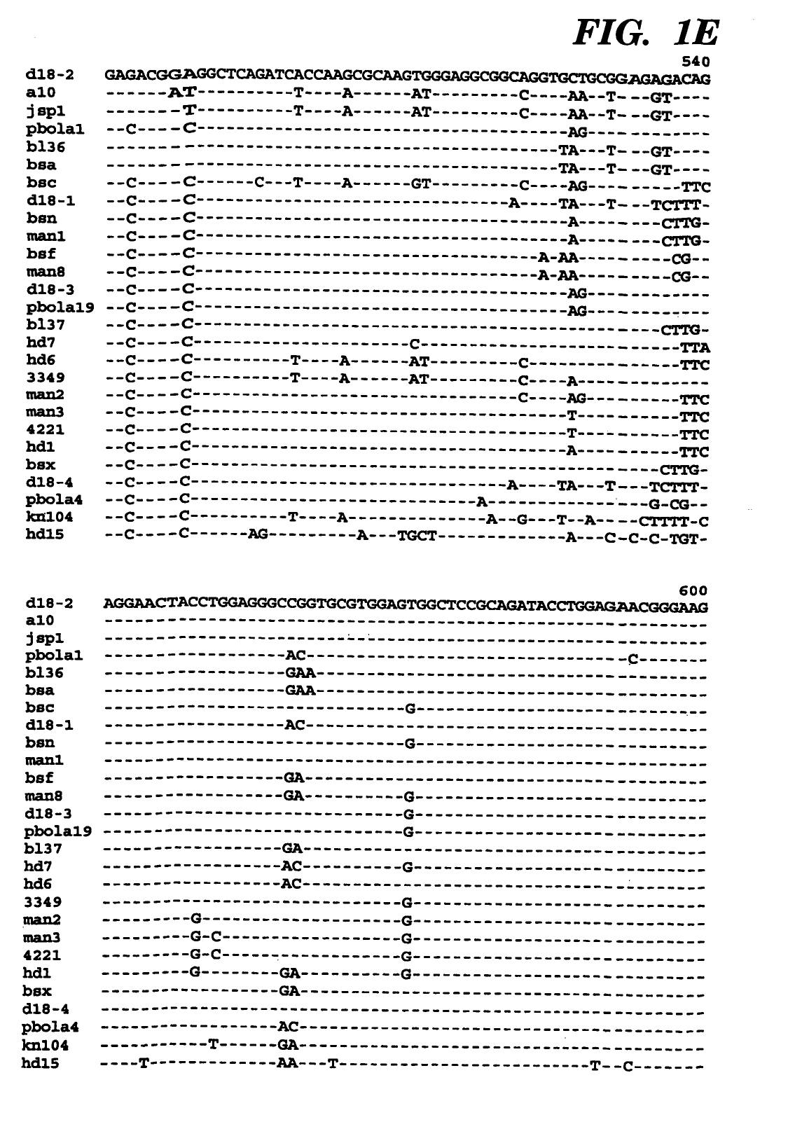

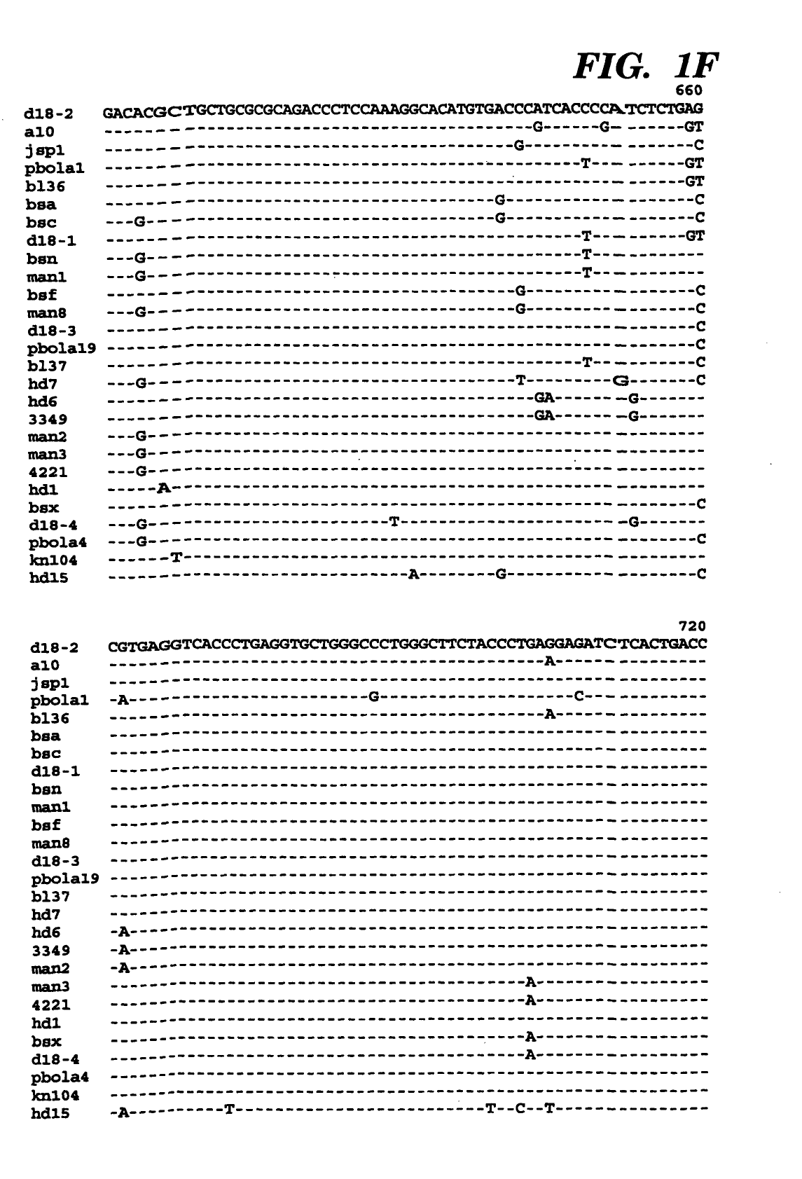

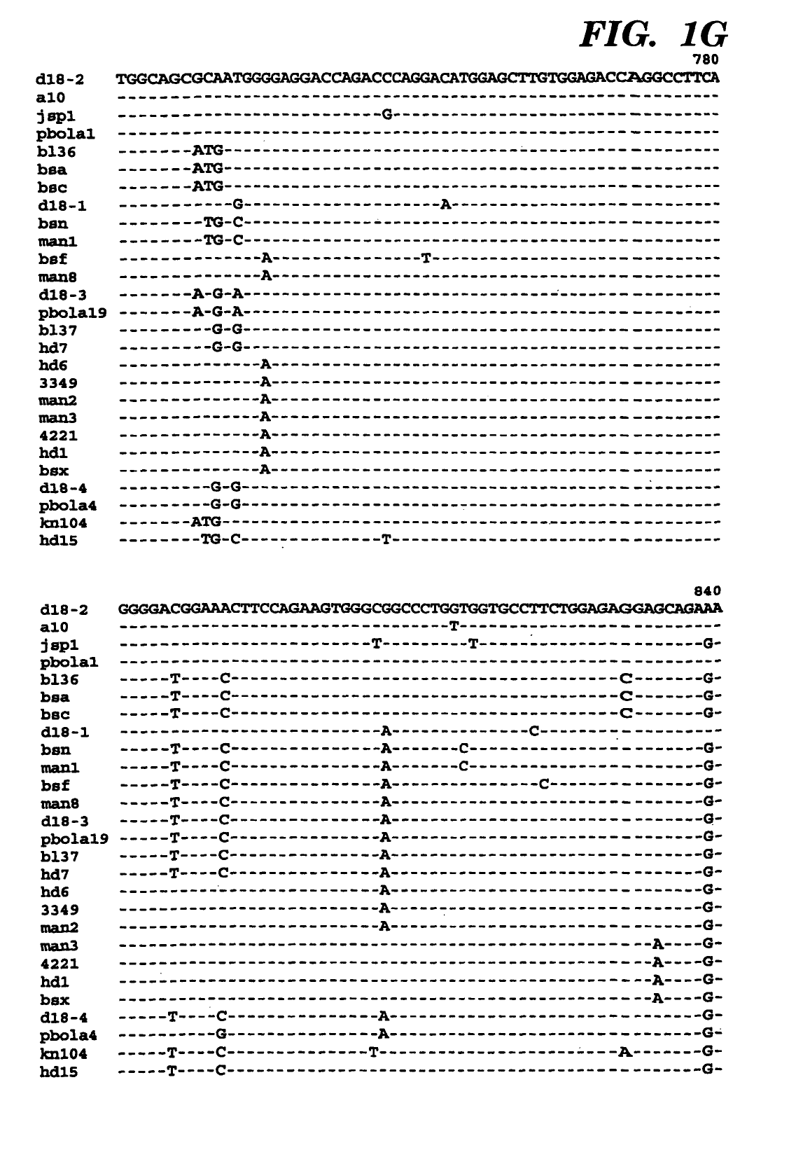

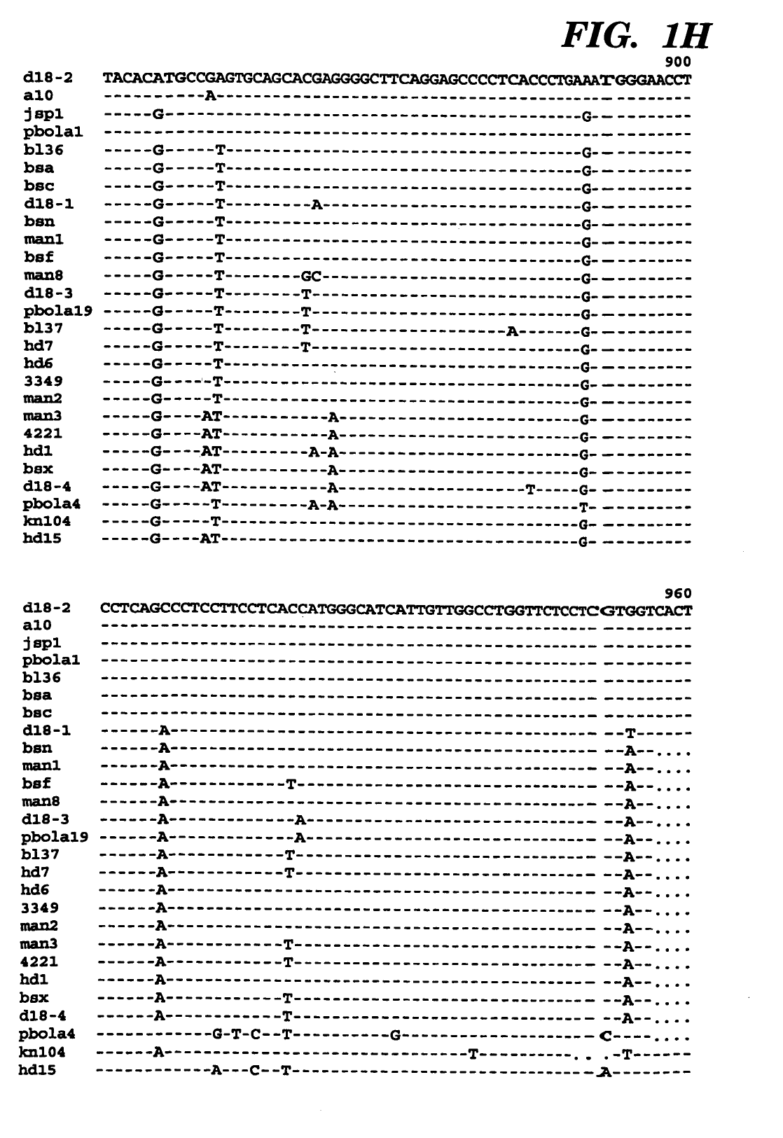

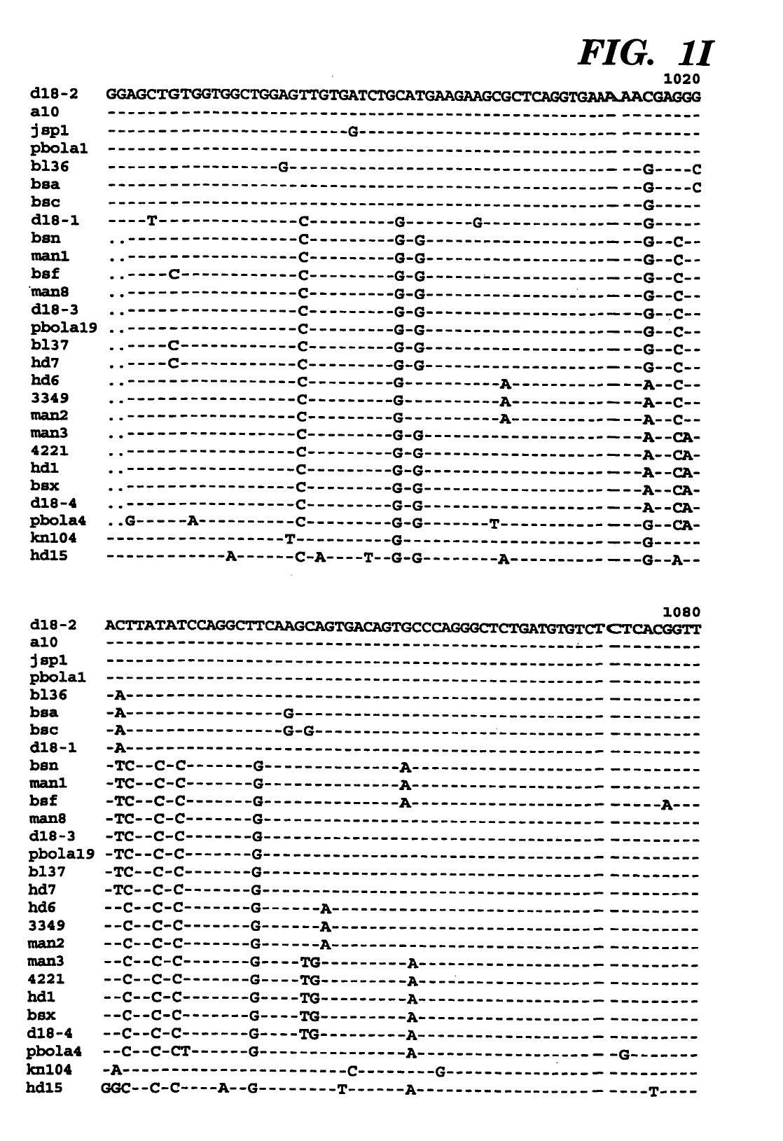

The determination of MHC-I antigen haplotype can be performed separately on individual NT embryos or it can be performed on a number of NT embryos in a single screening event. The same is true for making the determination of MHC-I antigen haplotype for the embryo recipients. A number of approaches can be utilized to perform the haplotyping, either alone or in combination. These include, without limitation, serological typing (Lewin 1996; Davies & Antczak 1991; Davies et al. 1994a); one dimensional-isoelectric focusing (Joosten et al. 1988; Davies et al. 1994a; Lewin 1996); DNA sequencing (Garber et al. 1993; Pichowski et al. 1996; Ellis et al. 1999); polymerase chain reaction amplification using allele specific primers (Ellis et al. 1998); and polymorphism analysis using oligonucleotide probes (Davies et al. 2001). With respect to the use of oligonucleotide probes (Davies et al. 2001), hybridization arrays can be created with probes that are specific to a number of different MHC-I genes and the resulting hybridization array patterns can be analyzed using computer software, e.g. the Cytofile genotyping software (Davies 1988). FIGS. 1A-J illustrate a nucleotide sequence alignment for a number of known MHC-I alleles. Probes can be selected based on the polymorphism which exists among the various MHC-I alleles. Haplotype assignments for the NT embryo and the embryo recipient can be based on one or more of these methods.

For the polymorphism analysis, an MHC-I microarray typing system can be used. This typing system includes a substrate and a plurality of oligonucleotide probes bound to the substrate, each of the plurality of oligonucleotides binding to at least one MHC-I allele, wherein each MHC-I allele binds to different oligonucleotide probes (i.e., a different subset of oligonucleotide probes).

According to a second embodiment, the method for minimizing immunological rejection of an NT fetus involves transferring, into an embryo recipient, an NT embryo having an MHC-I antigen type which is incompatible with an MHC-I antigen type of the embryo recipient.

A first approach to minimize immunological rejection in this situation, a involves down-regulation of MHC-I expression in the placenta of the NT embryo or fetus. Down-regulation of MHC-I expression by placental trophoblast cells is preferred, although down-regulation of MHC-I expression by other placental cells is also beneficial.

Down-regulation of MHC-I expression (in placental cells of NT embryos) can be achieved by (i) modulating expression of an MHC-I transcription factor in the NT embryo or fetus; (ii) treating the NT embryo or fetus with a cytokine, a growth factor, or combinations thereof which is suitable to inhibit MHC-I expression; or (iii) both (i) and (ii).

Without being bound by theory, it is believed that the regulation of MHC-I genes in bovine trophoblast cells may involve many of the same positive regulatory elements as human MHC-I genes (Harms L, Splitter 1994; Harms et al. 1995; Barker et al. 1997). In humans, down regulation of expression of “classical” MHC-I antigens on trophoblast cells involves both the absence of key transcription factors (CIITA and NF-κB/Rel family members p50 and p65) and the presence of specific negative regulatory factors (Gobin & van den Elsen 1999, 2000; Chiang & Main 1994; Coady et al. 1999; Peyman 1999). Introduction of a transgene expressing the RNA suppressor element (TSU) described by Peyman (1999) would be one option for the down regulation of MHC-I expression in the trophoblast cells of NT embryos. TSU is a particularly good candidate as an homologous goat expressed sequence tag (EST) was described in the original paper.

When treating the NT embryo or fetus with a cytokine or growth factor, the treatment can be carried out prior to transfer (i.e., in vitro), after transfer (i.e., in utero), or both. For in vitro treatment, a suitable cytokine or growth factor is introduced into the growth medium in which the NT embryo resides following nuclear transfer, such as the above-described medium utilized for activation. For in vivo treatment, a suitable cytokine or growth factor can be administered via intrauterine delivery or intravenous injection.

Suitable cytokines that can be employed to down-regulate MHC-I expression levels include, without limitation, several interleukins such as IL4, IL-10 and IL-13, leukemia inhibitory factor (“LIF”) and transforming growth factor-β (“TGF-β”), which has both cytokine and growth factor activities, or combinations thereof (Mitchell et al. 1993; Robertson et al. 1994; Moreau et al. 1999). While IL-10 can directly down-regulate MHC-I expression (see Moreau et al. 1999), it is believed that the other cytokines act indirectly by inhibiting the production of inflammatory cytokines (particularly INF-gamma) that induce MHC-I expression.

Suitable growth factors that can also be employed to down-regulate MHC-I expression levels include, without limitation, insulin, epidermal growth factor (“EGF”), granulocyte/macrophage colony-stimulating factor (“GM-CSF”), TGF-β, insulin-like growth factor(s) (“IGFs”), interleukin-3 (“IL-3”), or combinations thereof (Mitchell el al. 1993; Robertson et al. 1994).

A second approach to minimize immunological rejection in this situation involves suppressing an immune response of the embryo recipient. Suppression of the embryo recipient's immune response to the MHC-I incompatible embryo or fetus is effected by administering an amount of an immunosuppresive drug to the embryo recipient under conditions effective to suppress the anti-MHC-I immune response. Suitable immunosuppressive drugs include, without limitation, cyclosporin A, tacrolimus, and sirolimus. These exemplary immunosuppressive drugs are believed to cause immunosuppression by blocking signaling pathways in lymphocytes, thereby blocking immunological rejection. These immunosuppressive drugs can be administered systemically (i.e., intravenous) to the embryo recipient.

These two approaches for minimizing immunological rejection in MHC-I incompatible NT pregnancies can be utilized alone or in combination.

### EXAMPLES

The following examples are intended to illustrate, but by no means are intended to limit, the scope of the present invention as set forth in the appended claims.

Materials & Methods

### Leukocyte Immunohistochemistry

Immunoperoxidase staining for leukocyte differentiation antigens was performed on 8 μm sections of frozen uterine and placental tissues. For each pregnancy, sections from a minimum of two placentomal and two interplacentomal blocks were assessed. Staining was performed using the three-stage avidin-biotin system described under the SBU3 staining. The following antibodies can be used: anti-CD2 (mAb CC42; BioSource), CD3 (mAb MM1A; VMRD), CD4 (mAb CC30; BioSource), CD8β (mAb CC58; Serotec), TCR-γ/δ (mAb GB21A; VMRD), CD21 (mAb CC21; Serotec), CD25 (IL-2 receptor, mAb CACT 116A, VMRD), CD68 (mAb EMB11; DAKO), and MHC class II (mAb H42A; VMRD). Antigen positive cells in the placentomal and interplacentomal endometrium are enumerated by digital image processing with NIH Image software (Grünig et al. 1995).

Cytokine Immunohistochemistry

Immunohistochemistry can be used to compare cytokine production between groups and to identify cytokine producing cells at tore uterine/placental interface. For each pregnancy, sections from at least two placentomal and two interplacentomal blocks would be assessed. Staining can be done using the three-stage avidin-biotin system described above. Antibodies against the following cytokines can be used: IL-2 (mAb 14.1, VMRD), IL-4 (mAb CC303, Serotec; Weynants et al. 1998), IL-10 (goat anti-human IL-i 0, R & D Systems; Brown et al. 1994), IL-12 (mAb CC301, Serotec), IFN-γ (mAb CC302, Serotec), TNF-α (mAb 2C4-1D3 and polyclonal rabbit anti-bovine TNF-α, generously provided by Dr. Ted Elsasser; Palmer et al. 1998; Kenison et al. 1990; Sileghem et al. 1992), TGFβ1 and TGFβ2 (rabbit anti-human TGFβ1 and TGFβ2 from R & D Systems; Munson et al. 1996), and GM-CSF (mAb CC305, Serotec). Normal mouse ascites, rabbit serum or goat serum can be used as a negative control. Identification of cytokine positive cells can be based on cell location and morphologic features. The leukocyte differentiation antigen immunohistochemistry described above would be invaluable in the interpretation of the cytokine immunohistochemistry. The number of positive cells and the intensity of staining would be assessed using digital image analysis with NIH Image software (Grünig et al. 1995).

### Example 1

### Microarray MHC-I Typing

A bovine MHC-I microarray typing system was prepared by providing 17-22 bp oligonucleotides spotted on epoxy-silane treated, 12-well, Teflon masked, glass slides (Erie Scientific) using an Affymetrix 417 arrayer (Call et al. 2001). The MHC-I typing array is based on 118 known cDNA or genomic sequences from the BoLA Nomenclature Web Site and GenBank. As shown in Tables 1-4 below, two series of exon 2 probes and two series of exon 3 probes are provided. The exon 2 probes include 25 series A probes for codons 61-68 and 30 series B probes for codons 71-78 (see also FIGS. 1A-J). The exon 3 probes include 27 series A probes for codons 111-118 and 31 series B probes for codons 151-158 (see also FIGS. 1A-J). Together, these probes (and the corresponding polymorphisms) define an undetermined number of MHC-I haplotypes.

TABLE 1

BoLA Glass I, Exon 2, Series A Probes

| | | | | | |
|---|---|---|---|---|---|
| ID | | | # | | GC |
| No | Oligo Name | Sequence | bp | TM | % |

| | | | | | |
|---|---|---|---|---|---|
| 33 | BoLA-C1Ex2A01 | CGGGAGACGCAAAGGGCC | 18 | 57 | 72 |
| 34 | BoLA-C1Ex2A02 | CAGGAGACGCGAAAGGCC | 18 | 55 | 67 |
| 35 | BoLA-C1Ex2A03 | CGGAACACGCGAAACGCG | 18 | 55 | 67 |
| 36 | BoLA-C1Ex2A04L | ATCGAAACACGAGAATCTACAA | 22 | 49 | 36 |
| 37 | BoLA-C1Ex2A05 | GAGCAGACGCGAATAGTC | 18 | 50 | 56 |
| 38 | BoLA-C1Ex2A06 | CGCGAGACGCGAAACTCC | 18 | 55 | 67 |
| 39 | BoLA-C1Ex2A07 | CGCGAGACTCAAATCTCC | 18 | 50 | 56 |
| 40 | BoLA-C1Ex2A08 | CGCGAGACGCGAATCTCC | 18 | 55 | 67 |
| 41 | BoLA-C1Ex2A09 | CAGAACACGCGAAACTCC | 18 | 50 | 56 |
| 42 | BoLA-C1Ex2A10 | GAGGAGACGTGGAGAGCC | 18 | 55 | 67 |
| 43 | BoLA-C1Ex2A11 | CAGGAGACGCAGAGAACT | 18 | 50 | 56 |
| 44 | BoLA-C1Ex2A12 | CAAGAGACGCGGATACAA | 18 | 48 | 50 |
| 45 | BoLA-C1Ex2A13 | CAGGCGACGCAGAGAACT | 18 | 53 | 61 |
| 46 | BoLA-C1Ex2A14 | CAGGAGACGCGAAACGCC | 18 | 55 | 67 |
| 47 | BoLA-C1Ex2A15 | GAGATGACACGAGATGCC | 18 | 50 | 56 |
| 48 | BoLA-C1Ex2A17 | GACGAGACGCGAATCTCC | 18 | 53 | 61 |
| 49 | BoLA-C1Ex2A18R | CGGTGTGCTTGAAGTTTCGC | 20 | 54 | 55 |
| 50 | BoLA-C1Ex2A19R | CGGCGGCCTTTAAGTTTCG | 19 | 53 | 58 |
| 51 | BoLA-C1Ex2A20 | GCGATGACAAGAGATGCC | 18 | 50 | 56 |
| 52 | BoLA-C1Ex2A21 | CAGAACACGCGAAACGCC | 18 | 53 | 61 |
| 53 | BoLA-C1Ex2A22 | CACTGAGACGCAGAGGACT | 18 | 53 | 61 |
| 54 | BoLA-C1Ex2A23L | CATCAGGAGACGCAGATAACT | 21 | 52 | 48 |
| 55 | BoLA-C1Ex2A25 | GAGGAAACGCAAAGGGGC | 18 | 53 | 61 |
| 56 | BoLA-C1Ex2A26 | GAGGAGACGCAAAGGGCC | 18 | 55 | 67 |
| 57 | BoLA-C1Ex2A27 | TCGAAACACGAGGATCTACA | 20 | 50 | 45 |

TABLE 2

BoLA Class I, Exon 2, Series B Probes

| | | | | | |
|---|---|---|---|---|---|
| ID | | | # | | GC |
| No | Oligo Name | Sequence | bp | TM | % |

| | | | | | |
|---|---|---|---|---|---|
| 58 | BoLA-C1Ex2B01L | CAGATTTTCCGAGTGAGCC | 19 | 51 | 53 |
| 59 | BoLA-C1Ex2B02L | CAATTTTTCCGAGTGAGCCT | 20 | 50 | 45 |
| 60 | BoLA-CIEx2B03 | CAGACTTTCCGGGCGAAC | 18 | 53 | 61 |
| 61 | BoLA-C1Ex2B04L | CAGAGTTTCCGAGTGAACCT | 20 | 52 | 50 |
| 62 | BoLA-C1Ex2B0SL | CAGACTTTCCGAGTGGACC | 19 | 53 | 58 |
| 63 | BoLA-C1Ex2B07 | CAGACTTTCCGAGCGAAC | 18 | 50 | 56 |
| 64 | BoLA-C1Ex2B08L | CAGACTTTCCGAGTGTACC | 19 | 51 | 53 |
| 65 | BoLA-C1Ex2B09 | CAGATTTTCCGGGCGAAC | 18 | 50 | 56 |
| 66 | BoLA-C1Ex2B10L | CAGATTTTCCGAGTGGACC | 19 | 51 | 53 |
| 67 | BoLA-C1Ex2B11 | CAGTCTTTCCGAGTGGGC | 18 | 53 | 61 |
| 68 | BoLA-C1Ex2B12 | CTGTGGTACCGAGAGGCC | 18 | 55 | 67 |
| 69 | BoLA-C1Ex2B13 | CTGGTGTATCGAGGGAGC | 18 | 53 | 61 |
| 70 | BoLA-C1Ex2B14L | ACTGGTGTATCGAGAGAGC | 19 | 51 | 53 |
| 71 | BoLA-C1Ex2B15L | CTGGTATATCGAGAGAGCC | 19 | 51 | 53 |
| 72 | BoLA-C1Ex2B16L | CAATTTTTCCGAGGGGGCC | 19 | 53 | 58 |
| 73 | BoLA-C1Ex2B17L | ACAATTTTTCCGAGTGTAGCT | 21 | 49 | 38 |
| 74 | BoLA-C1Ex2B18L | CAGAATTTCCGAGTGGGCC | 19 | 53 | 58 |
| 75 | BoLA-C1Ex2B19 | CAGACTTTCCGAGCAAAC | 18 | 48 | 50 |
| 76 | BoLA-C1Ex2B22L | CTGCTGTATCGAGAGAACC | 19 | 51 | 53 |
| 77 | BoLA-C1Ex2B23 | CTGAAGTACCGAGAGGCC | 18 | 53 | 61 |
| 78 | BoLA-C1Ex2B24L | CAGAAATCCCGATTATGCTTG | 21 | 50 | 43 |
| 79 | BoLA-C1Ex2B25L | CAGGAATCCCGATTATGCTT | 20 | 50 | 45 |
| 80 | BoLA-C1Ex2B26L2 | CTGCTGTATCGAAAGAACCT | 20 | 50 | 45 |
| 81 | BoLA-C1Ex2B27 | CAGAGATTGCGAACGGGC | 18 | 53 | 61 |
| 82 | BoLA-C1Ex2B28L | CAGACTTTCCGAGTGAACC | 19 | 51 | 53 |
| 83 | BoLA-C1Ex2B29L | CAGAGATCCGAATTATGCTTG | 21 | 50 | 43 |
| 84 | BoLA-C1Ex2B30L | CAGTCTTTCCGAGTGAACC | 19 | 51 | 53 |
| 85 | BoLA-C1Ex2B31L | CAGTTTCGGAGTGAACCTGA | 20 | 52 | 50 |
| 86 | BoLA-C1Ex2B32L | CAGGTTTTCCAAGTGAAGCT | 20 | 50 | 45 |
| 87 | BoLA-C1Ex2B33L | CAGGTTTTCCGAGTGAACC | 19 | 51 | 53 |

TABLE 3

BoLA Class I, Exon 3, Series A Probes

| | | | | | |
|---|---|---|---|---|---|
| ID | | | # | | GC |
| No | Oligo Name | Sequence | bp | TM | % |

| | | | | | |
|---|---|---|---|---|---|
| 88 | BoLA-C1Ex3A01R | GGCGTCCTGCCTGTATCC | 18 | 55 | 67 |
| 89 | BoLA-C1Ex3A02R | GCCGAACTGCTCATAGCC | 18 | 53 | 61 |
| 90 | BoLA-C1Ex3A03R | GGCGTTCTGCCAGATCCC | 18 | 55 | 67 |
| 91 | BoLA-C1Ex3A04R | GGCGTCCTGCCTGTACC | 17 | 54 | 71 |
| 92 | BoLA-C1Ex3A05R | AGCGTCCTGCCTGTACCC | 18 | 55 | 67 |
| 93 | BoLA-C1Ex3A06R | GGCGTACTGCCTGTACCC | 18 | 55 | 67 |
| 94 | BoLA-C1Ex3A07R | GGCGTCCTGACTGTACCC | 18 | 55 | 67 |
| 95 | BoLA-C1Ex3A08R | GGCGAACTGATCGTACCC | 18 | 53 | 61 |
| 96 | BoLA-C1Ex3A09R | GGCGAGCTGATTATACCCG | 19 | 53 | 58 |
| 97 | BoLA-C1Ex3A10R | GGCGTCCTGATTATACCCG | 19 | 53 | 58 |
| 98 | BoLA-C1Ex3A11R | GCCGAACTGCGTATACCC | 18 | 53 | 61 |
| 99 | BoLA-C1Ex3A12R | GGCGTCCTGCTCATACCC | 18 | 55 | 67 |
| 100 | BoLA-C1Ex3A13R | GCCGTACTGCTGATACCC | 18 | 53 | 61 |
| 101 | BoLA-C1Ex3A14R | GCCGTACTGATCATACCCG | 19 | 53 | 58 |
| 102 | BoLA-C1Ex3A15R | GGCTAACTGATCATACCCG | 19 | 51 | 53 |
| 103 | BoLA-C1Ex3A16R | GGCGAACTGATCATACCCG | 19 | 53 | 58 |
| 104 | BoLA-C1Ex3A17R | GCCGTACTGCTAATACCCG | 19 | 53 | 58 |
| 105 | BoLA-C1Ex3A18R | GGCGAACTGCTTGAACCC | 18 | 53 | 61 |
| 106 | BoLA-C1Ex3A19R | GCCGAACTGCGTGAACCC | 18 | 55 | 67 |
| 107 | BoLA-C1Ex3A20R | GGCGTCCTGCATGAACCC | 18 | 55 | 67 |
| 108 | BoLA-C1Ex3A21R | GCCGTACTGCATGAACCC | 18 | 53 | 61 |
| 109 | BoLA-C1Ex3A22R | GGCGAACTGCATGAACCC | 18 | 53 | 61 |
| 110 | BoLA-C1Ex3A23R | GCCGAACTGCATGAACCC | 18 | 53 | 61 |
| 111 | BoLA-C1Ex3A24R | GCCGAACTGCCAGAACCC | 18 | 55 | 67 |
| 112 | BoLA-CIEx3A25R | GCCGAACTGCCAAAACCC | 18 | 53 | 61 |
| 113 | BoLA-C1Ex3A26R | GGCGAAGTGATCATAGCGC | 19 | 53 | 58 |
| 114 | BoLA-C1Ex3A27R | GGCCTTCTGCCAGAATCCA | 19 | 53 | 58 |

TABLE 4

BoLA Class I, Exon 3, Series B Probes

| | | | | | |
|---|---|---|---|---|---|
| ID | | | # | | GC |
| No | Oligo Name | Sequence | bp | TM | % |

| | | | | | |
|---|---|---|---|---|---|
| 115 | BoLA-C1Ex3B01 | GGCTGAGGAGAGACAGAC | 18 | 53 | 61 |
| 116 | BoLA-C1Ex3B06L | GGCAGGGAAAGATCCAACG | 19 | 53 | 58 |
| 117 | BoLA-C1Ex3B07 | GGAGGCAGAGTTCCAACG | 18 | 53 | 61 |
| 118 | BoLA-C1Ex3B08 | TAATGCGGAGAGCGAGAG | 18 | 50 | 56 |
| 119 | BoLA-C1Ex3B09 | TAATGCGGAGAGCGGGAG | 18 | 53 | 61 |
| 120 | BoLA-C1Ex3B10 | TCGTGCGGAGAGATTCAG | 18 | 50 | 56 |
| 121 | BoLA-C1Ex3B11L | GTGAAGCTGAGGTACAGAG | 19 | 51 | 53 |
| 122 | BoLA-C1Ex3B12N | TGAGGCGGAGAGAGACAG | 18 | 53 | 61 |
| 123 | BoLA-C1Ex3B13 | TGAGGCGGAGAGACGCAG | 18 | 55 | 67 |
| 124 | BoLA-C1Ex3B15 | TGAGGCGGAGAGATTCAG | 18 | 50 | 56 |
| 125 | BoLA-C1Ex3B16 | TGATGCCGCGCGTGTGAG | 18 | 55 | 67 |
| 126 | BoLA-C1Ex3B17L | GTGATGCGGAGACTTGGAG | 19 | 53 | 58 |
| 127 | BoLA-C1Ex3B18L | GTGATGCGGAGAGACAGAG | 19 | 53 | 58 |
| 128 | BoLA-C1Ex3B19L | GGTGATGCGGAGAGATTAAG | 20 | 52 | 50 |
| 129 | BoLA-C1Ex3B20L | GTGATGCGGAGAGATTCAG | 19 | 51 | 53 |
| 130 | BoLA-C1Ex3B21 | TGATGCGGAGGGACACAG | 18 | 53 | 61 |
| 131 | BoLA-C1Ex3B22 | TGATGCGGGGCGTGTGAG | 18 | 55 | 67 |
| 132 | BoLA-C1Ex3B23 | TGCTGCGAAGGGCGAGAG | 18 | 55 | 67 |
| 133 | BoLA-C1Ex3B24 | TGCTGCGGAGACTTGGAG | 18 | 53 | 61 |
| 134 | BoLA-C1Ex3B25 | TGCTGCGGAGAGACAGAG | 18 | 53 | 61 |
| 135 | BoLA-C1Ex3B26L | GTGCTGCGGAGAGATTAAG | 19 | 51 | 53 |
| 136 | BoLA-C1Ex3B27 | TGCTGCGGAGAGATTCAG | 18 | 50 | 56 |
| 137 | BoLA-C1Ex3B28 | TGCTGCGGAGCGTGTGAG | 18 | 55 | 67 |
| 138 | BoLA-C1Ex3B29S | TGCTGCGGAGGGCGAGA | 17 | 54 | 71 |
| 139 | BoLA-C1Ex3B30L | GTGTTGCGGAGAGATTCAG | 19 | 51 | 53 |
| 140 | BoLA-C1Ex3B31L | GTTACGCTGAGGTACAGAG | 19 | 51 | 53 |
| 141 | BoLA-C1Ex3B32L | GTTATGCTGAGGTACAGAG | 19 | 49 | 47 |
| 142 | BoLA-C1Ex3B33L | CAGATTATGGTGAGTCTTTGA | 21 | 49 | 38 |
| 143 | BoLA-C1Ex3B34L | GGTTCTACGGACTTTTACAG | 20 | 50 | 45 |
| 144 | BoLA-C1Ex3B35 | TTCTGCGGAGAGCGGGAG | 18 | 55 | 67 |
| 145 | BoLA-C1Ex3B38L | AAGGTTATGCTGAGTCTTTGA | 21 | 49 | 38 |

The nucleotide sequences of MHC-I alleles appearing in the following Genbank Accession Nos. were used to prepare the probes listed in Tables 1-4 above: M69204, AB008573-AB008654 inclusive, AB009347-AB009349 inclusive, AB009359, AB009360, AB009655, AB013099, AJ010861-AJ010867 inclusive, AJ271292, AJ271294, L02832-L02835 inclusive, M21043, M21044, M24090, M69206, U01187, X80933-X80936 inclusive, X82672, X92870, X97645, and Y09205-Y09208 inclusive. (In addition to the above-reported nucleotide sequences, probes for these same regions can be utilized for any new alleles identified hereafter.)

A hemi-nested PCR protocol was used to amply exons 2 and 3 together from genomic DNA (primers BoC1FP-E2A/E2B and BoC1RP-E3C) followed by amplification of each exon independently. For second stage amplifications biotinylated forward and reverse primers are be used to amplify each exon. The primer sequences are as follows:

Class I exon 2

| | |
|---|---|
| mixture of BoC1FP-E2A/E2B | (SEQ ID Nos: 28, 29) |

| | |
|---|---|
| acgtggacga cacg(c/g)agttc | 20 |

| | |
|---|---|
| and BoC1RP-E2A | (SEQ ID No: 30) |

| | |
|---|---|
| ctcgctctgg ttgtagtagc c | 21 |

| | |
|---|---|
| Class I exon 3 | |
| BoC1FP-E3D | (SEQ ID No: 31) |

| | |
|---|---|
| tggtcggggc gggtcagggt ctcac | 25 |

| | |
|---|---|
| and BoC1RP-E3C | (SEQ ID No: 32) |

| | |
|---|---|
| ccttcccgtt ctccaggtat ctgcggagc | 29 |

Following 10 cycles of first round PCR amplification and 35 cycles of second round amplification, 20 μl of the 25 μl PCR reaction is diluted to 100 μl with blocking buffer (150 mM Na-Citrate, 5× Denharts), denatured for 5 minutes at 95° C., and 35 μl of diluted PCR product hybridized to a well of a corresponding microarray slide overnight at 50° C. Slides are washed in room temperature, 0.1×SSPE, incubated for 1 hour at room temperature with 35 μl Streptavidin-Alexa Fluor® 546 conjugate (Molecular Probes) diluted 1:500 in blocking buffer, rinsed in 0.1×SSPE, dried and scanned on an Applied Precision ArrayWoRx scanner. Spots are scored on a 5-point scale from negative to strongly positive and data is interpreted using Cytofile genotyping software (Davies 1988; Davies et al. 1994b).

### Example 2

### Nuclear Transfer

Cryopreserved aliquots of cell suspensions from a Nellore fetus removed by hysterotomy at Day 45 of gestation were used to provide donor cells. The donor cells were derived from cells frozen at passage 2 (Day 10 of culture), then thawed and cultured in 4 well Nunc plates containing Dulbecco's Modified Eagles medium (DMEM-F12)+10% v:v fetal bovine serum (FBS)+1% v:v penicillin/streptomycin at 37° C. in air containing 5% CO₂. At 50% confluence they were serum starved (0.5% FBS) for 5 days prior to NT.

Recipient oocytes were slaughterhouse derived and matured for 17 hours in Medium 199 (M 199; Gibco Laboratories Inc.; Grand Island, N.Y.) supplemented with 10% v:v fetal calf serum (FCS; Gibco), FSH 0.1 units/ml (Sioux Biochem; Sioux City, Iowa), LH 0.1 units/ml (Sioux Biochem), estradiol 1 μg/ml (Sigma; St Louis, Mo.), 0.1 mM Cysteamine (Sigma M 9768), and 1% penicillin-streptomycin. The cumulus-oocyte complexes were vortexed 17 hours post maturation for 3 min in 0.1% hyaluronidase, washed, and then held in M 199+4 mg/ml BSA.

Oocytes were enucleated beginning at 19 h post maturation. Prior to enucleation, oocytes were placed for 15 min in Hepes-buffered M199 containing Hanks salts (H-M199; Gibco) with 4 mg/ml fatty acid free BSA (Sigma) plus 7.5 μg/ml cytochalasin B (Sigma) and 5 μg/ml Hoechst 33342 (Sigma). Oocytes were selected for the presence of a polar body and homogeneous cytoplasm. Suitable oocytes were enucleated in H-M199 with 7.5 μg/ml cytochalasin B using a beveled 25 μm outside diameter glass pipette. Only oocytes in which the removal of both the polar body and metaphase nucleus was confirmed by observation under UV light were included in the experiment.

Fibroblasts were combined with enucleated oocytes in H-M199 using a 25 μm outside diameter glass pipette, then returned to M199+4 mg/ml BSA. The oocyte-fibroblast couplets were manually aligned with a mouth pipette in groups of 4-6 and fused in a 0.5 mm fusion chamber (BTX) that contained mannitol 270 mM and magnesium chloride 0.05 mM (Wells & Powell 2000). Fusion parameters were 1×40 μsec 2.25 kV/cm DC fusion pulses delivered by a BTX Electrocell Manipulator 830 (BTX; San Diego, Calif.). Oocyte-fibroblast fusion was assessed 20-30 minutes later by light microscopy and unfused couplets were refused. Oocyte activation were performed 3-5 h after fusion at 27 h post maturation, by a 4 min incubation in Hepes buffered M199+5 μM ionomycin (Calbiochem; San Diego, Calif.), then 4 minutes in 30 mg/ml H199+BSA followed by washing in 4 mg/ml BSA in H-M199. The fused oocytes were transferred into 2 mM DMAP in M199+3 mg/ml BSA for 4 h followed by transfer to the embryo culture medium for 7 days. Embryos were cultured in 50:1 drops of a derivative of synthetic oviductal fluid serum-free medium (BARC-1; Wells and Powell, 2000) under mineral oil (Sage Biopharma, Bedminster, N.J.) in a 5% CO₂, 5% O₂, 90% N₂ atmosphere.

Cloned embryos classified as Grade 1 or 2 blastocysts on Day 6 following NT were transferred. Two blastocysts were non-surgically transferred into each recipient at Day 6.5 after natural or induced heat. Recipient cows were evaluated for pregnancy at 21 days following NT (15 days after embryo transfer) by serum progesterone levels and the first direct pregnancy examination was by transrectal ultrasonography at Day 32 following NT. Fetuses with a detectable heartbeat were recovered following slaughter at Day 35.

### Example 3

### Comparison of MHC-1 Expression in Non-MHC-I Matched NT Fetuses and Control Fetuses

A fibroblast cell line was derived from an in vivo produced Day 45 Nellore fetus. To produce the fetus, three embryos recovered non-surgically from a donor cow were transferred the same day into three recipient cows, all of which were pregnant at Day 45. The Nellore cell line was selected with a goal of amplifying any differences that may arise between tissue types of the donor tissue (Bos indicus) and recipient cows (Bos taurus—Angus). Fetal fibroblasts were derived from passage 2 cells (10-15 days in culture) and serum starved for 5 days prior to NT. NT was performed as previously described (Hill et al. 2000a) except that embryos were cultured for 7 days in a defined serum-free medium (BARC-1; Wells & Powell 2000).

The development rate for cloned embryos to blastocyst prior to selection of embryos for transfer into recipient cows is shown in Table 5 below.

TABLE 5
Development rate of cloned embryos to blastocyst

| Cell | Oocytes | Percent | No. Blasts | Percent Blasts |
|---|---|---|---|---|
| Line | Enucleated | Fusion | (Day 8) | (Day 8) |
| N 737F | 312 | 78% | 86 | 35.4% |

Day 7 embryos were shipped in a temperature-controlled 39° C. incubator to a commercial embryo transfer center (Trans Ova, Iowa) for transfer into synchronous recipient cows. The per embryo survival rate to Day 35 was 23% when transferred in pairs and the recipient cow pregnancy rate was 50%. Six cloned fetuses were recovered from 5 recipient cows between Day 35-50 of gestation.

Tissue samples were collected within 30 minutes of slaughter. If feasible, separate placentomal and interplacentomal samples were collected. However, in the Day 35 placentas, distinction between cotyledonar, and intercotyledonary areas by visual inspection is difficult. Tissues were be fixed in 4% paraformaldehyde for histology and for immunohistochemistry by freezing in OCT freezing compound. Fetal heart, liver, lung, kidney, gut, and flank muscle were also processed for histology. For immunohistochemistry, 2×2.5 cm rectangular sections of apposed placenta and uterus would be excised, anchored in plastic boats with OCT, and immediately frozen in isopentane chilled in liquid nitrogen. Frozen tissues were held on dry ice and then transferred to a −80° C. freezer for storage. For sectioning, blocks were warmed to −30° C. and cryostat sectioned at 8 μm. Sections were transferred to slides, dried at room temperature for 30 minutes, fixed in cold acetone for 15 minutes, air dried for 30 minutes, and returned to the freezer for storage. If “normal” placentomes with villus crypt interdigitation, and “failing” placentomes, where attachment is not occurring, were present, at least two tissue blocks containing each type of placentome were collected.

Control tissues (Holstein origin) were collected from commercial dairy cows sent to slaughter at a commercial slaughterhouse (Taylor Packing, Pennsylvania). Tissues were collected and processed on site as described above. Pregnant tracts were initially selected for gestational age by palpation of amniotic vesicle. After opening the uterus, the crown rump length was measured and the fetal age determined using a formula developed for purebred Holsteins by Rexroad et al. (1974).

MHC-I immunocytochemistry was performed on frozen sections from 6 NT and 8 control placentas within the range of 35-55 days of gestation (see Tables 6 and 7 below) as previously described (Davies et al. 2000). Basically, cryostat sections were blocked with normal goat serum and incubated with a 1:6000 dilution of IL-A19 anti-bovine MHC-I mAb (Bensaid et al. 1989; generously provided by Jan Naessens, ILRI, Nairobi, Kenya) or control antibody for two hours at 37° C. Detection of antigen/antibody complexes were achieved using a three stage avidin-biotin system and the AEC chromogen. For each pregnancy a minimum of two interplacentomal and two placentomal sections were examined. If both “normal” and “failing” placentomes were present, at least two placentomes of each type were assessed. The percent of MHC-I positive trophoblast and maternal epithelium was determined by visual assessment of a minimum of ten 100× fields. A reticle was used to define a constant field length. To eliminate inter-operator error, a single investigator read all slides.

TABLE 6
MHC-I expression in the 6 cloned fetuses recovered from 5 recipient cows

| | | | | | | MHC-I | | MHC-I |
|---|---|---|---|---|---|---|---|---|
| | Fetal | | | | | % of total | | % of total |
| NT | Viabi- | | | Endometrial | MHC-I | trophoblast | MHC-I | endometrium |
| Fetus | lity | Age | CR | lymphocytes | Cotyledon | positive | Endometrium | positive |
| 1 | dead | 35 | 0.7 | +++++ | +++++ | 97 | ++++ | 74 |
| | | | | foci | | | | |
| 2 | live | 35 | 1.5 | +++++ | +++++ | 93 | ++ | 44 |
| | | | | foci | | | | |
| 3 | live | 35 | 1.3 | +++++ | +++ | 58 | +++ | 68 |
| | | | | foci | | | | |
| 4 | live | 40 | 1.7 | + | — | 0 | − | 10 |
| 5a | live | 50 | 4 | + | — | 0 | + | 15 |
| 5b | dead | 50 | 3.5 | + | — | 0 | + | 15 |

5a and 5b were twins.
Age of fetus calculated from known NT dates.

TABLE 7
MHC-I expression in 8 control fetuses recovered from 7 cows

| | | | | | | MHC-I | | MHC-I |
|---|---|---|---|---|---|---|---|---|
| con- | Fetal | | | | | % of total | | % of total |
| trol | Viabi- | | | Endometrial | MHC-I | trophoblast | MHC-I | endometrium |
| Fetus | lity | Age | CR | lymphocytes | Cotyledon | positive | Endometrium | positive |
| 1a | Dead | 39 | 2 | + | − | 0 | + | 20 |
| 1b | Live | 39 | 2 | + | − | 0 | + | 20 |
| 2 | Live | 41 | 2.5 | ++ | − | 0 | ++ | 37 |
| 3 | Live | 45 | 3.5 | + | − | 0 | ++ | 35 |
| 4 | Live | 45 | 3.5 | + | − | 0 | + | 5 |
| 5 | Live | 45 | 3.5 | ++ | − | 0 | − | 0 |
| 6 | Live | 54 | 6 | + | − | 0 | + | 10 |
| 7 | Live | 54 | 6 | + | − | 0 | + | 5 |

Age of fetus calculated from the crown rump measurement using the formula of Rexroad et al. 1974.

Each of the 3 positive placentas was at 35 days of gestation while the 3 negative placentas were at 40 or 50 days. Based on these results, fetuses that do not express MHC-I are able to develop more normal placentation and have a higher probability of reaching the 2nd trimester of pregnancy. Non-viable fetuses were present in the cloned group. Two of 6 cloned fetuses were non-viable (as determined by lack of heartbeat on ultrasonographic scan on the previous day and confirmed by presence of amniotic hemorrhage at slaughter). One of these non-viable fetuses was MHC-I positive (Day 35 single) whereas the other was negative (a Day 50 twin).

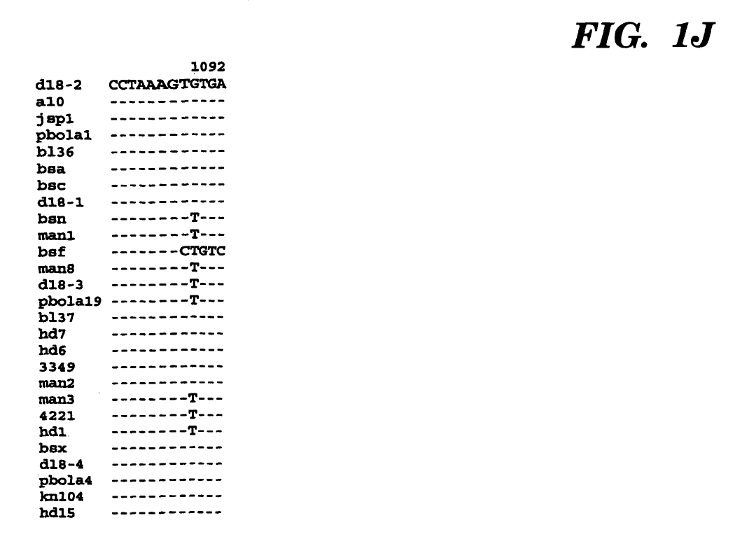

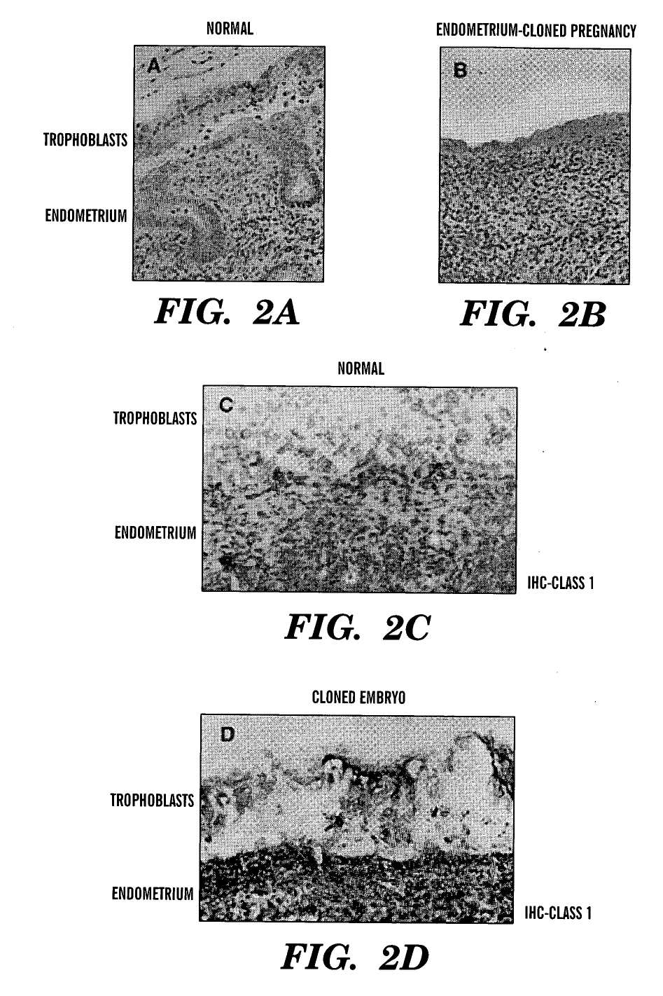

A striking feature of the endometrium of the recipient cows carrying the 3 cloned fetuses with MHC-I positive trophoblast was widespread endometrial inflammation. There were multiple foci of stromal lymphocyte and plasma cell accumulations in the caruncular and intercaruncular areas (compare FIGS. 2A-B). These areas were not subtle accumulations, but were instead strikingly obvious even at low power on hematoxylin and eosin (“H&E”) sections. The degree of lymphocytic infiltrate was similar for each of the 3 MHC-I positive pregnancies. Additionally, no neutrophils were found that would indicate an infectious endometritis. This degree of lymphocytic infiltration was not present in the caruncles of the MHC-I negative cloned placentas or of the 8 control placentas. Some areas of minor lymphocyte accumulations were found in the stratum compactum. These accumulations were deeper and more segmental than the widespread, commonly focal sub-epithelial accumulations in the MHC-I positive group.

CD3 immunostaining of endometrial sections from cloned and control pregnancies confirmed the H&E diagnosis that these cells were indeed lymphocytes. In the three initial Day 35 cloned pregnancies recovered (Table 6: NT fetuses 1, 2 and 3), far greater numbers of lymphocytes were apparent on histological examination of multiple fields from multiple sections. The most striking observation was that of lymphocyte (cd3 positive) aggregates in the stratum compactum of the intercotyledonary areas of endometrium. Interspersed between these aggregates were increased numbers of lymphocytes and plasma cells mainly distributed immediately beneath the epithelium and adjacent to the endometrial glands. Aggregates were defined as areas of cd3 positive cells where more than 20 cells were in contact with each other. Objective counts of numbers of aggregates and interaggregate cd3 positive lymphocytes were determined by visual estimation using a 0.292 mm² reticle to delineate linear boundaries per field. Mean counts per field were totaled per section, and means per case were calculated. A minimum of 5 fields per section, 4 sections from interplacentomal tissues, per case, was scored.

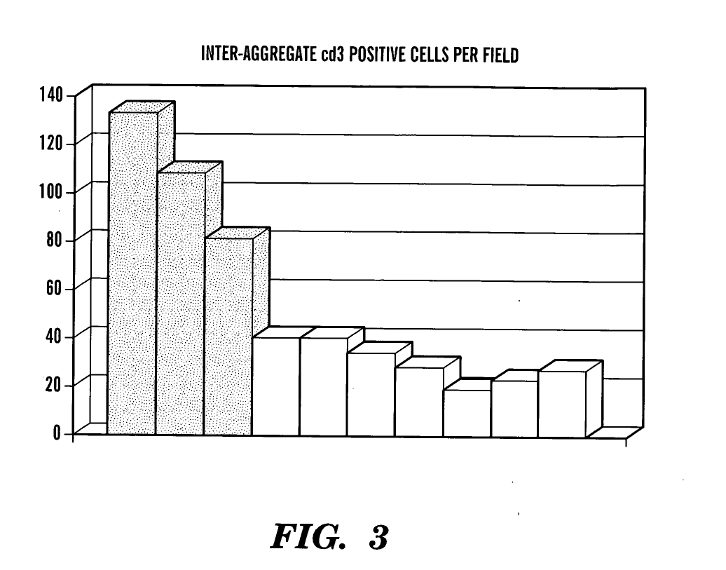

The cd3 positive aggregates were rare in the seven controls (4/158; 0.03% of fields), but found in over half the fields examined in the three clones (39.62; 62.9% of fields, p<0.001, Chi-square test). The mean number of aggregates per field was thus significantly higher in clones than controls (0.639 ∀0.09 vs 0.025 ∀0.012; p<0.001, Mann-Whitney rank sum test). These aggregates contained hundreds of cd3 positive lymphocytes in cross section. As illustrated in FIG. 3, cd3 positive lymphocytes located away from these aggregates (interaggregate cd3 positive cells) were also found to be significantly higher in the cloned pregnancies (p<0.001). Thus, the combined numbers of cd3 positive cells (aggregate+interaggregate) in the endometrium of cloned pregnancies were far higher than in controls (p<0.001; One Way Anova with Tukey pairwise comparisons).

The possibility that the observed endometrial inflammatory reaction in the cloned pregnancies (i.e., elevated cd3 positive cells) is caused by fetal death is unlikely when the cd3 numbers are compared from the one dead clone and one dead control fetus in this data set. The dead clone had the highest number of cd3 positive aggregates (0.8 aggregates per field) and interaggregate cd3 cells (133 ∀38 cells per field; bar 1 in FIG. 3) whereas the dead control fetus had no aggregates and a normal number of interaggregate cells (28 ∀ V 7 cells per field; bar 10 in FIG. 3). The crown rump length for the dead clone was less than half that expected for a Day 35 fetus (0.7 cm vs expected of 1.9 cm). This indicated either failure of fetal development or a hostile uterine environment.

While lymphocytic infiltration in the uterus of the non-viable fetus may logically be explained by release of fetal antigens to the endometrium, no signs of inflammation were present in endometrium of the other non-viable fetus—the Day 50 MHC-I negative clone. Thus, trophoblast MHC-I expression correlated with endometrial lymphocytic accumulations.

This small group of clones provides compelling evidence that a substantial proportion of the high early embryonic mortality observed in cloned pregnancies is due to inappropriate trophoblast MHC-I expression and immunologically mediated placental rejection.

Moreover, there is a remarkable similarity between the placental characteristics from NT and interspecies ET fetuses such as horse/donkey ((Allen, 1982) and sheep/goat (Hancock et al., 1968; Hancock. McGovern, 1970). We previously detailed an 82% loss rate in first trimester cloned fetuses, reduced placental vascularity, and rudimentary implantation sites (Hill et al. 2000b). Similar observations have been recorded in interspecies ET from donkeys into horses, where 80% (20/22) of first trimester fetuses failed by Day 90 and implantation sites were abnormal, as demonstrated above. The gross vascularity of the interspecies placentas was reduced, villous and crypt formation was rudimentary and there was widespread accumulation of lymphocytes in the endometrium. It was also determined that the only donkey foal that progressed to term possessed the same tissue type (MHC-I) as the recipient mare. In goat/sheep embryo transfers, placental attachment either failed to be established or to be maintained (Hancock et al., 1968; Hancock, McGovern, 1970). Underdeveloped cotyledons and lack of villous formation were characteristic findings and the histological findings were suggestive of maternal immune rejection of the placental tissue (Dent et al., 1971). These observations detail intriguing similarities in placental pathology between interspecies and NT fetuses.

In most mammals, trophoblast cells do not express major histocompatibility antigens. Davies et al. (2000) demonstrated trophoblast expression of MHC-I antigens, which first appeared during the sixth month of pregnancy and was limited to the interplacentomal and placentomal arcade regions, with no expression in the placentomal villus/crypt region. This region is the area of intimate fetal-maternal contact and it suggests that down regulation of MHC-I is necessary to avoid immunological rejection.

### LIST OF REFERENCES

Each of the references cited in the present application or otherwise listed below is hereby incorporated by reference in its entirety into the specification of this application.

Alexander B M, Johnson M S, Guardia R O, Graaf WLvd, Senger P L, Sasser R G, 1995. Embryonic loss from 30 to 60 days post breeding and the effect of palpation per rectum on pregnancy. Theriogenology 43:551-556.

Allen W R, 1982. Immunological aspects of the endometrial cup reaction and the effect of xenogeneic pregnancy in horses and donkeys. J. Reprod. Fertil. Suppl. 31:57-94:57-94.

Baguisi A, Behboodi E, Melican D T, Pollock J S, Destrempes M M, Cammuso C, Williams J L, Nims S D, Porter C A, Midura P, Palacios M J, Ayres S L, Denniston R S, Hayes M L, Ziomek C A, Meade H M, Godke R A, Gavin W G, Overstrom E W, Echelard Y, 1999. Production of goats by somatic cell nuclear transfer. Nat. Biotechnol. 17:456-461.

Barker N, Young J R, Morrison W I, Ellis S A, 1997. Sequence diversity present within the 5′ upstream regions of BoLA class I genes. Immunogenetics, 46:352-354.

Bensaid A, Kaushal A, MacHugh N D, Shapiro S Z, Teale A J, 1989. Biochemical characterization of activation-associated bovine class I major histocompatibility complex antigens. Anim. Genet. 20:241-255.

Brown W C, Woods V M, Chitko-McKown C G, Hash S M, Rice-Ficht A C, 1994. Interleukin-10 is expressed by bovine type 1 helper, type 2 helper, and unrestricted parasite-specific T-cell clones and inhibits proliferation of all three subsets in an accessory-cell-dependent manner. Infection and Immunity 62:4697-4708.

Call D R, Chandler D P, Brockman F, 2001. Fabrication of DNA microarrays using unmodified oligonucleotide probes. BioTechniques 30(2):368-372. 374. 376.

Campbell K H, McWhir J, Ritchie W A, Wilmut I, 1996. Sheep cloned by nuclear transfer from a cultured cell line. Nature 380:64-66.

Chiang M H, Main E K, 1994. Nuclear regulation of HLA class I genes in human trophoblasts. Am. J. Reprod. Immunol., 32:167-172.

Coady M A, Mandapati D, Arunachalam B, Jensen K, Maher S E, Bothwell A L, Hammond G L, 1999. Dominant negative suppression of major histocompatibility complex genes occurs in trophoblasts. Transplantation, 67:1461-1467.

Cibelli J B, Stice S L, Golueke P J, Kane J J, Jerry J, Blackwell C, Ponce de Leon F A, Robl J M, 1998. Cloned transgenic calves produced from nonquiescent fetal fibroblasts. Science 280:1256-1258.

Collas P, Barnes F L, 1994. Nuclear transplantation by microinjection of inner cell mass and granulosa cell nuclei. Mol. Reprod. Dev. 38:264-267.

Collas P, Robl J M, 1990. Factors affecting the efficiency of nuclear transplantation in the rabbit embryo. Biol. Reprod. 43:877-884.

Cuthbertson K S, Cobbold P H, 1985. Phorbol ester and sperm activate mouse oocytes by inducing sustained oscillations in cell Ca²⁺. Nature 316:541-542.

Davies C J, 1988. Immunogenetic characterization of the class II region of the bovine major histocompatibility complex. Ph.D. thesis, Cornell University, Ithaca, N.Y., University Microfilms International, Ann Arbor.

Davies C J, Antczak D F, 1991. Mixed lymphocyte culture studies reveal complexity in the bovine major histocompatibility complex not detected by class I serology. Anim. Genet. 22:31-44.

Davies C J, Joosten I, Bemoco D, Arriens M A, Bester J, Ceriotti G, Ellis S, Hensen E J, Hines H C, Horin P, Kristensen B, Lewin HA, Meggiolaro D, Morgan A L G, Morita M, Nilsson PhR, Oliver R A, Orlova A, Østerg\dot{a}rd H, Park C A, Schuberth H-J, Simon M, Spooner R L, Stewart J A, 1994a. Polymorphism of bovine MHC class I genes. Joint report of the Fifth International Bovine Lymphocyte Antigen (BoLA) Workshop, Interlaken, Switzerland, Aug. 1, 1992. Eur. J. Immunogenet. 21:239-258.

Davies C J, Joosten I, Andersson L, Arriens M A, Bernoco D, Bissumbhar B, Byrns G, van Eijk M J T, Kristensen B, Lewin H A, Mikko S, Morgan A L G, Muggli-Cockett N E, Nilsson PhR, Oliver R A, Park Calif., van der Poel J J, Polli M, Spooner R L, Stewart J A, 1994b. Polymorphism of bovine MHC class II genes. Joint report of the Fifth International Bovine Lymphocyte Antigen (BoLA) Workshop, Interlaken, Switzerland, Aug. 1, 1992. Eur. J. Immunogenet. 21:259-289.

Davies C J, Fisher P J, Schlafer D H, 2000. Temporal and regional regulation of major histocompatibility complex class I expression at the bovine uterine/placental interface. Placenta 21:194-202.

Davies C J, Reynolds J O, Call D R, 2001. Microarray based MHC typing for cattle. Proceedings of the Sixth International Veterinary Immunology Symposium, 218 (Abstract).

Dent J, McGovern P T, Hancock J L, 1971. Immunological implications of ultrastructural studies of goat X sheep hybrid placentae. Nature 231:116-117.

Ellis S A, Holmes E C, Staines K A, Smith K B, Stear M J, McKeever D J, MacHugh N D, Morrison W I, 1999. Variation in the number of expressed MHC genes in different cattle class I haplotypes. Immunogenetics 50:319-328.

Ellis S A, Staines K A, Stear M J, Hensen E J, Morrison W I, 1998. DNA typing for BoLA class I using sequence-specific primers, PCR-SSP. Eur. J. Immunogenet., 25:365-370.

Forar A L, Gay J M, Hancock D D, Gay C C, 1996. Fetal loss frequency in ten holstein dairy herds. Theriogenology 45:1505-1513.

Garber T L, Hughes A L, Letvin N L, Templeton J W, Watkins D I, 1993. Sequence and evolution of cattle MHC class I cDNAs: concerted evolution has not taken place in cattle. Immunogenetics, 38:11-20.

Gobin S J P, van den Elsen P J, 1999. The regulation of HLA class I expression: is HLA-G the odd one out? Cancer Biology, 9:55-59.

Gobin S J P, van den Elsen P J, 2000. Transcriptional regulation of the MHC class Ib genes HLA-E, HLA-F, and HLA-G. Hum. Immunol., 61:1102-1107.

Graham C F, 1969. The fusion of cells with one and two cell mouse embryos. Wister Inot. Symp. Monogr., 9:19.

Grinig G, Triplett L, Canady L K, Allen W R, Antczak D F, 1995. The maternal leucocyte response to the endometrial cups in horses is correlated with the developmental stages of the invasive trophoblast cells. Placenta 16:539-559.

Hancock J L, McGovern P T, Stamp J T, 1968. Failure of gestation of goat x sheep hybrids in goats and sheep. J. Reprod. Fertil. Suppl. Suppl 3:29-36.

Hancock J L, McGovern P T, 1970. Placentae of goat-sheep hybrids. J. Anat. 106:413.

Harms J S, Splitter G A, 1994. The enhancer A/IRS region of a cattle MHC class I promoter. Immunogenetics, 39:372.

Harms J S, Li W, Splitter G A, 1995. The cattle major histocompatibility complex, MHC. class-I possesses HLA-like promoters. Gene, 160:249-252.

Hasler J F, Henderson W B, Hurtgen P J, Jin Z Q, McCauley A D, Mower S A, Neely B, Shuey L S, Stokes J E, Trimmer S A, 1995. Production, freezing and transfer of bovine IVF embryos and subsequent calving results. Theriogenology 43:141-152.

Hill J R, Roussel A J, Cibelli J B, Edwards J F, Hooper R N, Miller M W, Thompson J A, Looney C R, Westhusin M E, Robl J M, Stice S L, 1999a. Clinical and pathologic features of cloned transgenic calves and fetuses (13 Case Studies). Theriogenology 51:1451-1465.

Hill J R, Winger Q A, Burghardt R C, Westhusin M E, 1999b. The effect of donor cell serum starvation and oocyte activation compounds on the development of somatic cell cloned embryos. Cloning 1:201-209.

Hill J R, Winger Q A, Long C R, Looney C R, Thompson J A, Westhusin M E, 2000a. Development rates of male bovine nuclear transfer embryos derived from adult and fetal cells. Biol. Reprod. 62(5):1135-40.

Hill J R, Burghardt R C, Jones K, Long C R, Looney C R, Shin T, Spencer T E, Thompson J A, Winger Q A, Westhusin M E, 2000b. Evidence for Placental Abnormality as the Major Cause of Mortality in First-Trimester Somatic Cell Cloned Bovine Fetuses. Biol. Reprod. 63:1787-1794.

Joosten I, Oliver R A, Spooner R L, Williams J L, Hepkema B G, Sanders M F, Hensen E J, 1988. Characterization of class I bovine lymphocyte antigens, BoLA. by one-dimensional isoelectric focusing. Anim. Genet., 19:103-113.

Kato Y, Tetsuya T, Sotomaru Y, Kurokawa K, Kato J, Doguchi H, Yasue H, Tsunoda Y, 1998. Eight calves cloned from somatic cells of a single adult. Science 282:2095-2098.

Kato Y, Tani T, Tsunoda Y, 2000. Cloning of calves from various somatic cell types of male and female adult, newborn and fetal cows. J. Reprod. Fertil. 120 (2):231-237.

Kenison D C, Elsasser T H, Fayer R, 1990. Radioimmunoassay for bovine tumor necrosis factor: concentration and circulating molecular forms in bovine plasma. J. Immunoassay 11: 177-198.

King G J, Atkinson B A, Robertson H A, 1979. Development of the bovine placentome during the second month of gestation. J. Reprod. Fertil. 55:173-180.

Kubota C, Yamakuchi H, Todoroki J, Mizoshita K, Tabara N, Barber M, Yang X, 2000. Six cloned calves produced from adult fibroblast cells after long-term culture. Proc. Natl. Acad. Sci. USA 97(3):990-5

Lewin H A, 1996. Genetic Organization, polymorphism, and function of the bovine major histocompatability complex (Chapter 4). In Schook, L B and Lamont, S J (ed.) The Major Histocompatability Complex Region of Domestic Animals CRC Press, pp. 65-98.

Mitchell M D, Trautman M S, Dudley D J, 1993. Cytokine networking in the placenta. Placenta, 14:249-275.

Moreau P, Adrian-Cabestre F, Menier C, Guiard V, Gourand L, Dausset J, Carosella ED, Paul P, 2001. IL-10 selectively induces HLA-G expression in human trophoblasts and monocytes. International Immunology, 11:803-811.

Munson L, Wilhite A, Boltz V F, Wilkinson J E, 1996. Transforming growth factor beta in bovine placentas. Biol. Reprod. 55:748-755.

Noden D M, de Lahunta A, 1990. Extraembryonic membranes and placentation. In: Noden D M, de Lahunta A (eds.), The embryology of domestic animals. Baltimore: Williams and Wilkins, pp. 47-69.

Onishi A, Iwamoto M, Akita T, Mikawa S, Takeda K, Awata T, Hanada H, Perry A C, 2000. Pig cloning by microinjection of fetal fibroblast nuclei. Science 289:1188-90.

Overbergh L, Valckx D, Waer M, Mathieu C, 1998. Quantification of murine cytokine mRNAs using real time quantitative reverse transcriptase PCR. Cytokine 11:305-312.

Ozil J P, 1990. The parthenogenetic development of rabbit oocytes after repetitive pulsatile electrical stimulation. Development 109:117-127.

Palmer M V, Elsasser T H, Cheville N F, 1998. Tumor necrosis factor-α in pregnant cattle after intravenous or subcutaneous vaccination with Brucella abortus strain RB51. Am. J. Vet. Res. 59:153-156.

Peyman J A, 1999. Repression of major histocompatibility complex genes by a human trophoblast ribonucleic acid. Biol. Reprod., 60:23-31.

Pichowski J S, Ellis S A, Morrison W I, 1996. Sequence of two cattle MHC class I cDNAs associated with BoLA A10 specificity. Immunogenetics, 43:253-254.

Polejaeva I A, Chen S H, Vaught T D, Page R L, Mullins J, Ball S, Dai Y, Boone J, Walker S, Ayares D L, Colman A, Campbell K H, 2000. Cloned pigs produced by nuclear transfer from adult somatic cells. Nature 407:86-90.

Prather, R S, Eichen P A, Nicks D K, Peters M S, 1991. Artificial activation of porcine oocytes matured in vitro. Mol. Reprod. Dev. 28(4):405-409.

Renard J P, Chastant S, Chesne P, Richard C, Marchal J, Cordonnier N, Chavette P, Vignon X, 1999. Lymphoid hypoplasia and somatic cloning. The Lancet 353:1489-1491.

Rexroad C E, Jr., Casida L E, Tyler W J, 1974. Crown-rump length of fetuses in purebred Holstein-Friesian cows. J Dairy Sci 57:346-347.

Robertson S A, Seamark R F, Guilbert L J, Wegmann T G, 1994. The Role of Cytokines in Gestation. Critical Reviews in Immunology, 14:239-292.

Robt et al., 1992. In Proceedings from the Symposium on Cloning Mammals by Nuclear Transplantation, (Seidel ed.), Colorado State University, Ft. Collins, Colo., pp. 24-27.

Schena, M, 2000. Microarray biochip technology. Eaton Publishing, Natick, Mass.

Siedel, G E, Jr. 1981. Critical review of embryo transfer procedures with cattle. In Fertilization and Embryonic Development in Vitro, Mastroianni L, Jr. and Biggers, J D, (eds), Plenum Press, New York, N.Y., page 323

Sileghem M, Saya R, Ellis J A, Flynn J N, Peel J E, Williams D J, 1992. Detection and neutralization of bovine tumor necrosis factor by a monoclonal antibody. Hybridoma 11:617-27.

Schnieke A E, Kind A J, Ritchie W A, Mycock K, Scott A R, Ritchie M, Wilmut I, Colman A, Campbell K H, 1997. Human factor IX transgenic sheep produced by transfer of nuclei from transfected fetal fibroblasts. Science 278:2130-2133.

Stice S L, Strelchenko N S, Keefer C L, Matthews L, 1996. Pluripotent bovine embryonic cell lines direct embryonic development following nuclear transfer. Biol. Reprod. 54: 100-110.

Thompson J G, Gardner D K, Pugh P A, McMillan W H, Tervit H R, 1995. Lamb birth weight is affected by culture system utilized during in vitro pre-elongation development of ovine embryos. Biol. Reprod. 53:1385-1391.

Vignon X, Chesne P, Le Bourhis D, Flechon J E, Heyman Y, Renard J P, 1998. Developmental potential of bovine embryos reconstructed from enucleated matured oocytes fused with cultured somatic cells. C.R. Acad. Sci. III 321:735-745.

Wakayama T, Perry A C, Zuccotti M, Johnson K R, Yanagimachi R, 1998. Full-term development of mice from enucleated oocytes injected with cumulus cell nuclei. Nature 394:369-374.

Wakayama T Yanagimachi R, 1999. Cloning of male mice from adult tail-tip cells. Nat. Genet. 22:127-128.

Walker S K, Hartwich K M, Seamark R F, 1996. The production of unusually large offspring following embryo manipulation—concepts and challenges. Theriogenology 45:111-120.

Wells D N, Misica P M, Day T A, Tervit H R, 1997. Production of cloned lambs from an established embryonic cell line: a comparison between in vivo- and in vitro-matured cytoplasts. Biol. Reprod. 57:385-393.

Wells D N, Pavla P M, Tervit H R, Vivanco W H, 1998. Adult somatic cell nuclear transfer is used to preserve the last surviving cow of the Enderby Island cattle breed. Reprod. Fertil. Dev. 10:369-378.

Wells D N, Misica P M, Tervit H R, 1999. Production of Cloned Calves Following Nuclear Transfer with Cultured Adult Mural Granulosa Cells. Biol. Reprod. 60:996-1005.

Wells K D, Powell A M, 2000. Blastomeres from somatic cell nuclear transfer embryos are not allocated randomly in chimeric blastocysts. Cloning 2:9-21.

Weynants V, Gilson D, Furger A, Collins R A, Mertens P, De Bolle X, Heussler V T, Roditi I, Howard C J, Dobbelaere A E, Letesson J J, 1998. Production and characterization of monoclonal antibodies specific for bovine interleukin-4. Vet. Immunol. Immunopath. 66:99-112.

Wilmut I, Sales D I, Ashworth C J, 1986. Maternal and embryonic factors associated with prenatal loss in mammals. J. Reprod. Fertil. 76:851-864.

Wilmut I, Schnieke A E, McWhir J, Kind K L, Campbell K H S, 1997. Viable offspring derived from fetal and adult mammalian cells. Nature 385:810-813.

Wooding F B P, Flint A P F, 1994. Placentation. In: Lamming GE (ed.), Marshall's Physiology of Reproduction. London: Chapman & Hall, pp. 233-429.

Young L E, Sinclair K D, Wilmut I, 1998. Large offspring syndrome in cattle and sheep. Rev. Reprod. 3:155-163.

U.S. Pat. No. 6,147,276 to Campbell et al.

U.S. Pat. No. 6,235,970 to Stice et al.

U.S. Pat. No. 5,496,720 to Susko-Parrish et al.

U.S. Pat. No. 5,057,420 to Massey

U.S. Pat. No. 4,994,384 to Prather et al.

PCT Publication No. WO 96/07732 to Campbell et al.

PCT Publication No. WO 94/26884 to Wheeler et al.

PCT Publication No. WO 94/24274 to Smith et al.

PCT Publication No. WO 90/03432 to Evans et al.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of minimizing immunological rejection of a nuclear transfer fetus comprising:
transferring a nuclear transfer embryo into an embryo recipient under conditions effective for development of a nuclear transfer fetus with minimal risk of immunological rejection of the fetus due to a maternal anti-fetal MHC-I immune response.

2. The method according to claim 1, wherein the nuclear transfer embryo comprises an MHC-I antigen type which is compatible with an MHC-I antigen type of the embryo recipient.

3. The method according to claim 2, further comprising:
determining the MHC-I antigen type for both the nuclear transfer embryo and the embryo recipient and
matching the nuclear transfer embryo suitable for transfer into the embryo recipient based on the MHC-I antigen types thereof.

4. The method according to claim 1, wherein the nuclear transfer embryo comprises an MHC-I antigen type which is incompatible with an MHC-I antigen type of the embryo recipient.

5. The method according to claim 4, further comprising:
regulating MHC-I expression of the nuclear transfer embryo or nuclear transfer fetus.

6. The method according to claim 5, wherein said regulating comprises:
modulating expression of an MHC-I transcription factor in the nuclear transfer embryo or nuclear transfer fetus.

7. The method according to claim 5, wherein said regulating comprises:
treating the nuclear transfer embryo or nuclear transfer fetus with a cytokine, a growth factor, or combinations thereof, under conditions effective to inhibit MHC-I expression.

8. The method according to claim 7, wherein said treating is carried out in vitro on the nuclear transfer embryo.

9. The method according to claim 7, wherein said treating is carried out in utero on the nuclear transfer embryo or the nuclear transfer fetus.

10. The method according to claim 7, wherein said treating is carried out with a cytokine.

11. The method according to claim 10, wherein the cytokine is IL-4, IL-10, IL-13, LIF, TGF-β, or combinations thereof.

12. The method according to claim 7, wherein said treating is carried out with a growth factor.

13. The method according to claim 12, wherein the growth factor is insulin, EGF, GM-CSF, TGF-β, IGF, IL-3, or combinations thereof.

14. The method according to claim 4, further comprising:
suppressing an immune response of the embryo recipient.

15. The method according to claim 14, wherein said suppressing comprises:
administering an amount of an immunosuppresive drug to the embryo recipient under conditions effective to suppress the anti-MHC-I immune response.

16. The method according to claim 15, wherein the immunosuppressive drug is cyclosporin A, tacrolimus, or sirolimus.

17. The method according to claim 1, wherein the embryo recipient is a mammal.

18. The method according to claim 17, wherein the mammal is a ruminant.

19. The method according to claim 1, wherein the nuclear transfer embryo is developed from non-human mammalian cells.

20. The method according to claim 19, wherein the non-human mammalian cells are ruminant cells.

21. The method according to claim 1, wherein said transferring is carried out by introducing the nuclear transfer embryo into the uterus of the embryo recipient.

22. A method of performing embryo transfer comprising:
determining an MHC-I antigen type for a nuclear transfer embryo and an MHC-I antigen type for embryo recipients and either
(i) transferring the nuclear transfer embryo into a first embryo recipient having a compatible MHC-I antigen type under conditions effective for development of a nuclear transfer fetus from the nuclear transfer embryo, or
(ii) transferring the nuclear transfer embryo into a second embryo recipient having an incompatible MHC-I antigen type and (a) regulating MHC-I expression of the nuclear transfer embryo or (b) suppressing an immune response of the embryo recipient, under conditions effective for development of a nuclear transfer fetus from the nuclear transfer embryo.

23. The method according to claim 22, wherein said transferring according to step (i) or step (ii) comprises implanting the nuclear transfer embryo in a uterus of the first or second embryo recipient.

24. The method according to claim 22, said method comprising transferring according to step (ii) and regulating MHC-I expression of the nuclear transfer embryo according to step (a).

25. The method according to claim 24, wherein said regulating comprises:
modulating expression of an MHC-I transcription factor in the nuclear transfer embryo.

26. The method according to claim 24, wherein said regulating comprises:
treating the nuclear transfer embryo with a cytokine. a growth factor, or combinations thereof, under conditions effective to inhibit MHC-I expression.

27. The method according to claim 26, wherein said treating is carried out in vitro on the nuclear transfer embryo.

28. The method according to claim 26, wherein said treating is carried out in utero on the nuclear transfer embryo.

29. The method according to claim 26, wherein said treating is carried out with a cytokine.

30. The method according to claim 29, wherein the cytokine is IL-4, IL-10, IL-13, LIF, TGF-β, or combinations thereof.

31. The method according to claim 26, wherein said treating is carried out with a growth factor.

32. The method according to claim 31, wherein the growth factor is insulin, EGF, GM-CSF, TGF-β, IGF, IL-3, or combinations thereof.

33. The method according to claim 24 further comprising:
regulating MHC-I expression of the nuclear transfer fetus under conditions effective for continued development of the nuclear transfer fetus.

34. The method according to claim 33, wherein said regulating comprises:
modulating expression of an MHC-I transcription factor in the nuclear transfer fetus.

35. The method according to claim 34, wherein said regulating comprises:
treating the nuclear transfer fetus with a cytokine, a growth factor, or combinations thereof, under conditions effective to inhibit MHC-I expression.

36. The method according to claim 35, wherein said treating is carried out in utero on the nuclear transfer fetus.

37. The method according to claim 35, wherein said treating is carried out with a cytokine.

38. The method according to claim 37, wherein the cytokine is IL-4, IL-10, IL-13, LIF, TGF-β, or combinations thereof.

39. The method according to claim 35, wherein said treating is carried out with a growth factor.

40. The method according to claim 39, wherein the growth factor is insulin, EGF, GM-CSF, TGF-β, IGF, IL-3, or combinations thereof.

41. The method according to claim 22, said method comprising transferring according to step (ii) and suppressing an immune response of the embryo recipient according to step (b).

42. The method according to claim 41, wherein said suppressing comprises:
administering an amount of an immunosuppresive drug to the embryo recipient under conditions effective to suppress the anti-MHC-I immune response.

43. The method according to claim 42, wherein the immunosuppressive drug is cyclosporin A, tacrolimus, or sirolimus.

44. The method according to claim 22, wherein the first or second embryo recipients is a mammal.

45. The method according to claim 44, wherein the mammal is a ruminant.

46. The method according to claim 22, wherein the nuclear transfer embryo is developed from non-human mammalian cells.

47. The method according to claim 46, wherein the non-human mammalian cells are ruminant cells.

48. An MHC-I microarray typing system comprising:
a substrate and
a plurality of oligonucleotide probes bound to the substrate, each of the plurality of oligonucleotides binding to at least one MHC-I allele, wherein each MHC-I allele binds to different oligonucleotide probes.

49. The MHC-I microarray typing system according to claim 48, wherein the at least one MHC-I allele is a bovine allele.

50. The MHC-I microarray typing system according to claim 49, wherein the plurality of oligonucleotide probes comprise:
a first set of oligonucleotide probes specific for MHC-I exon 2, codons 61-68;
a second set of oligonucleotide probes specific for MHC-I exon 2, codons 71-78;
a third set of oligonucleotide probes specific for MHC-I exon 3, codons 111-118; and
a fourth set of oligonucleotide probes specific for MHC-I exon 3, codons 151-158.


SEQUENCE LISTING (145 sequences)

SEQ ID NO: 1 - DNA, 1092 bp, Bos taurus
atgcgagttatggggccgcgaaccctcctcctgctgctctcgggggtcctggtcctgaccgagacccgggctggctcccactcgatgaggtatttcagcaccgccgtgtcccggcccggcctcggggagccccggtacctggaagtcggctacgtggacgacacgcagttcgtgcggttcgacagcgacgcccggaatccgaggatggagccgcgggaacggtgggtggagcaggaggggccggagtattgggatcgggagacgcaaagggccaagggcaacgcacagattttccgagtgagcctgaacaacctgcgcggctactacaaccagagcgaggccgggtctcacaccttccagtggatgtacggctgcgacgtggggccggacgggcgcctcctcggcgggtatgagcagtacggctacgacggcagagattacatcgccctgaacgaggacctgcgctcctggaccgcgggggagacggaggctcagatcaccaagcgcaagtgggaggcggcaggtgctgcggagagacagaggaactacctggagggccggtgcgtggagtggctccgcagatacctggagaacgggaaggacacgctgctgcgcgcagaccctccaaaggcacatgtgacccatcaccccatctctgagcgtgaggtcaccctgaggtgctgggccctgggcttctaccctgaggagatctcactgacctggcagcgcaatggggaggaccagacccaggacatggagcttgtggagaccaggccttcaggggacggaaacttccagaagtgggcggccctggtggtgccttctggagaggagcagaaatacacatgccgagtgcagcacgaggggcttcaggagcccctcaccctgaaatgggaacctcctcagccctccttcctcaccatgggcatcattgttggcctggttctcctcgtggtcactggagctgtggtggctggagttgtgatctgcatgaagaagcgctcaggtgaaaaacgagggacttatatccaggcttcaagcagtgacagtgcccagggctctgatgtgtctctcacggttcctaaagtgtga

SEQ ID NO: 2 - DNA, 1064 bp, Bos indicus
tcctgctgctctcgggggtcctggtcctgaccgagacccgggctggctcccactcgatgaggtatttcagcaccgccgtgtcccggcccggcctcggggagccccggtacctggaagtcggctacgtggacgacacgcagttcgtgcggtttgacagcgacgccccgaatccgaggatggagccgcgggcgcggtgggtggagcaggaggggccggagtattgggatcgggagacgcaaagggccaagggcaacgcacaatttttccgagtgagcctgaacaacctgcgcggctactacaaccagagcgaggccgggtctcacaccctccagtggatgtccggctgctacgtggggccggacgggcgtcctccgcgcgggttcatgcagttcggctacgacggcagagattacctcgccctgaacgaggacctgcgctcctggaccgcggtggagacgatggctcagatctccaaacgcaagatggaggcggccggtgaagctgaggtacagaggaactacctggagggccggtgcgtggagtggctccgcagatacctggagaacgggaaggacacgctgctgcgcgcagaccctccaaaggcacatgtgacccgtcacccgatctctggtcgtgaggtcaccctgaggtgctgggccctgggcttctaccctgaagagatctcactgacctggcagcgcaatggggaggaccagacccaggacatggagcttgtggagaccaggccttcaggggacggaaacttccagaagtgggcggccctgttggtgccttctggagaggagcagaaatacacatgccaagtgcagcacgaggggcttcaggagcccctcaccctgaaatgggaacctcctcagccctccttcctcaccatgggcatcattgttggcctggttctcctcgtggtcactggagctgtggtggctggagttgtgatctgcatgaagaagcgctcaggtgaaaaacgagggacttatatccaggcttcaagcagtgacagtgcccagggctctgatgtgtctctcacggttcctaaagtgtga

SEQ ID NO: 3 - DNA, 1069 bp, Bos taurus
cctcctcctgctgctctcgggggtcctggtcctgaccgagaccgtggcgggctcccactcgatgaggtatttcctcaccgccgtgtcccggcccggcttcggggagccccggtacctggaagtcggctacgtggacgacacgcagttcgtgcggttcgacagcgacgccccgaatccgaggatggagccgcgggcgcggtgggtggagcaggaggggccggagtattgggatcaggagacgcgaaaggccaagggcaacgcacaatttttccgagtgagcctgaacaacctgcgcggctactacaaccagagcgaggccgggtctcacaccctccagctgatgtccggctgctacgtggggccggacgggcgtctccgccgcgggttcatgcagttcggctacgacggcagagattacctcgccctgaacgaggacctgcgctcctggaccgcggtggagacggtggctcagatctccaaacgcaagatggaggcggccggtgaagctgaggtacagaggaactacctggagggccggtgcgtggagtggctccgcagatacctggagaacgggaaggacacgctgctgcgcgcagaccctccaaaggcacatgtgacgcatcaccccatctctgaccgtgaggtcaccctgaggtgctgggccctgggcttctaccctgaggagatctcactgacctggcagcgcaatggggaggaccagacgcaggacatggagcttgtggagaccaggccttcaggggacggaaacttccagaagtgggtggccctggttgtgccttctggagaggagcagagatacacgtgccgagtgcagcacgaggggcttcaggagcccctcaccctgagatgggaacctcctcagccctccttcctcaccatgggcatcattgttggcctggttctcctcgtggtcactggagctgtggtggctggagttgtggtctgcatgaagaagcgctcaggtgaaaaacgagggacttatatccaggcttcaagcagtgacagtgcccagggctctgatgtgtctctcacggttcctaaagtgtga

SEQ ID NO: 4 - DNA, 1090 bp, Bos taurus
gcctctgagtctgggaagaaacagagtcctgctgctctcgggggtcctggtcctgaccgagacccgggctggctcccactcgatgaggtatttcagcaccgccgtgtcccggcccggcctcggggagccccggtacctggaagtcggctacgtggacgacacgcagttcgtgcggttcgacagcgacgccccgaatccgaggatggagccgcgggcgcggtgggtggagcaggaggggcccgagtattgggatcaggagacgcgaaaggccaagggcaccgcacagactttccgggcgaacctgaacatcgcactcggctactacaaccagagcgaggccgggtctcacaccttccagtggatgtacggctgcgacgtggggccggacgggcgtctccgccgcgggttcatgcagtacggctacgacggcagagattacatcgccctgaacgaggacctgcgctcctggaccgcggcggacacggcggctcagatcaccaagcgcaagtgggaggcggcaggtgaggcggagagacagaggaactacctggagggcacgtgcgtggagtggctccgcagatacctggagaccgggaaggacacgctgctgcgcgcagaccctccaaaggcacatgtgacccatcactccatctctggtcatgaggtcaccctgaggtgctgggcgctgggcttctaccctgaggacatctcactgacctggcagcgcaatggggaggaccagacccaggacatggagcttgtggagaccaggccttcaggggacggaaacttccagaagtgggcggccctggtggtgccttctggagaggagcagaaatacacatgccgagtgcagcacgaggggcttcaggagcccctcaccctgaaatgggaacctcctcagccctccttcctcaccatgggcatcattgttggcctggttctcctcgtggtcactggagctgtggtggctggagttgtgatctgcatgaagaagcgctcaggtgaaaaacgagggacttatatccaggcttcaagcagtgacagtgcccagggctctgatgtgtctctcacggttcctaaagtgtga

SEQ ID NO: 5 - DNA, 1083 bp, Bos taurus
atggggccgcgagccctcctcctgctgctctcgggggtcctgatcctgactgagacccgggctggctcccactccctgaggtatttcagcaccgccgtgtcccggcccggcctcggggagccccggtacctggaagtcggctacgtggacgacacgcagttcgtgcagttcgacagcgacgccccgaatccgaggatggagccgcgggcgcggtgggtggagcaggaggggccggagtattgggatcggaacacgcgaaacgccaagggcaacgcacagagtttccgagtgaacctgaacaccctgcgcggctactacaaccagagcgaggccgggtctcacaccctccagtggatgtccggctgcgacgtggggccggacggggctctccgccgcgggttcatgcagtacggctacgacggtagagattacctcgccctgaacgaggacctgcgctcctggaccgcgggggagacggaggctcagatcaccaagcgcaagtgggaggcggcaggttatgctgaggtacagaggaactacctggagggcgaatgcgtggagtggctccgcagatacctggagaacgggaaggacacgctgctgcgcgcagaccctccaaaggcacatgtgacccatcaccccatctctggtcgtgaggtcaccctgaggtgctgggccctgggcttctaccctgaagagatctcactgacctggcagcatgatggggaggaccagacccaggacatggagcttgtggagaccaggccttcaggggatggaaccttccagaagtgggcggccctggtggtgccttctggagacgagcagagatacacgtgccgtgtgcagcacgaggggcttcaggagcccctcaccctgagatgggaacctcctcagccctccttcctcaccatgggcatcattgttggcctggttctcctcgtggtcactggagctgtggtggctggggttgtgatctgcatgaagaagcgctcaggtgaaaaaggaggcaattatatccaggcttcaagcagtgacagtgcccagggctctgatgtgtctctcacggttcctaaagtgtga

SEQ ID NO: 6 - DNA, 1083 bp, Bos taurus
atggggccgcgagccctcctcctgctgctctcgggggtcctggtcctgactgagacccgggctggctcccactccctgaggtatttcagcaccgccgtgtcccggcccggcttcggggagccccggtacctggaagtcggctacgtggacgacacgcagttcgtgcagttcgacagcgacgccccgaatccgaggatggagccgcgggcgcggtgggtggagcaggaggggccggagtattgggatcggaacacgcgaaacgccaagggcaacgcacagagtttccgagtgaacctgaacaccctgcgcggctactacaaccagagcgaggccgggtctcacaccctccagtggatgtctggctgcgacgtggggccggacgggcgtctccgccgcgggttcatgcagtacggctacgacggtagagattacctcgccctgaacgaggacctgcgctcctggaccgcgggggagacggaggctcagatcaccaagcgcaagtgggaggcggcaggttatgctgaggtacagaggaactacctggagggcgaatgcgtggagtggctccgcagatacctggagaacgggaaggacacgctgctgcgcgcagaccctccaaaggcacatgtggcccatcaccccatctctgaccgtgaggtcaccctgaggtgctgggccctgggcttctaccctgaggagatctcactgacctggcagcatgatggggaggaccagacccaggacatggagcttgtggagaccaggccttcaggggatggaaccttccagaagtgggcggccctggtggtgccttctggagacgagcagagatacacgtgccgtgtgcagcacgaggggcttcaggagcccctcaccctgagatgggaacctcctcagccctccttcctcaccatgggcatcattgttggcctggttctcctcgtggtcactggagctgtggtggctggagttgtgatctgcatgaagaagcgctcaggtgaaaaaggaggcaattatatccaggcttcaggcagtgacagtgcccagggctctgatgtgtctctcacggttcctaaagtgtga

SEQ ID NO: 7 - DNA, 1083 bp, Bos taurus
atggggccgcgagccctcctcctgctgctctcgggggtcttggtcctgaccgagacccgggcgggctcccactccctgaggtatttctacaccgccgtgtcccggcccggcctcggggagccccgcttcatctctgtcggctacgtggacaacacggagttcgtgcggttcgacagcgacgccccggatccgagggaagaaccgcgggtgaggtggatggagcaggaggggctggagtattgggatcgaaacacgagaatctacaaggacaccgcacagactttccgagtggacctgaacaccctgcgcggctactacaaccagagcgaggccgggtctcacaccctccaggagatgtacggctgcgacgtggggccggacgggcgcctcctcggcgggtatgagcagtacggctacgaaggcagagattacctcgccctgaacgaggacctgcgctcctggaccgcggcggacacggcggctcacatctccaaacgcaaggtggaggcggccggtgaggcggagagattcaggaactacctggagggccggtgcgtggaggggctccgcagatacctggagaacgggaaggacgcgctgctgcgcgcagaccctccaaaggcacatgtggcccatcaccccatctctgaccgtgaggtcaccctgaggtgctgggccctgggcttctaccctgaggagatctcactgacctggcagcatgatggggaggaccagacccaggacatggagcttgtggagaccaggccttcaggggatggaaccttccagaagtgggcggccctggtggtgccttctggagacgagcagagatacacgtgccgtgtgcagcacgaggggcttcaggagcccctcaccctgagatgggaacctcctcagccctccttcctcaccatgggcatcattgttggcctggttctcctcgtggtcactggagctgtggtggctggagttgtgatctgcatgaagaagcgctcaggtgaaaaaggagggaattatatccaggcttcagggagtgacagtgcccagggctctgatgtgtctctcacggttcctaaagtgtga

SEQ ID NO: 8 - DNA, 1083 bp, Bos taurus
atggggccgcgaaccctcctcctgctactctcgggggtcctggtcctgaccgagacccgggcaggctcccactccctgaggtatttcagcaccgccgtgtcccggcccggcctcgaggagccccgctttatcatcgtcggctacgtggacgacacgcagttcgtgcggttcgacagcgactccccgaatccgagggcagaaccgcgggcgccgtggatggagcaggaggggccggagtattgggatgagcagacgcgaatagtcaaggacaccgcacagactttccgggcgaacctgaacaccgcactcggctactacaaccagagcgaggccgggtctcacaatatccaggcgatgtacggctgcgacgtggggtcggacgggagtttcctccgcgggtacagtcaggacgcctacgacggcagagattacatcgccctgaacgaggacctgcgctcctggaccgcggcggacacggcggctcagatcaccaagcgcaagtgggaggcggaaggttatgctgagtctttgaggaactacctggagggcacgtgcgtggagtggctccgcagatacctggagaacgggaaggacacgctgctgcgcgcagaccctccaaaggcacatgtgacccatcactccatctctggtcgtgaggtcaccctgaggtgctgggccctgggcttctaccctgaggagatctcactgacctggcagcgcaagggggaggaccagacccaggaaatggagcttgtggagaccaggccttcaggggacggaaacttccagaagtgggcagccctggtggtgccctctggagaggagcagaaatacacgtgccgtgtgcagcacaaggggcttcaggagcccctcaccctgagatgggaacctcctcagacctccttcctcaccatgggcatcattgttggcctggttctcctcgttgtcactggagttgtggtggctggagctgtgatctggatgaagaggcgctcaggtgaaaaaggagggaattatatccaggcttcaagcagtgacagtgcccagggctctgatgtgtctctcacggttcctaaagtgtga

SEQ ID NO: 9 - DNA, 1059 bp, Bos taurus
atgctgctgctctcgggggtcctggtcctgaccgagaccctggcgggctcccactccctgaggtatttcctcaccgccgtgtcccggcccggcctcggggaaccccgcttcatcatcgtcggctacgtggacgacacgcagttcgtgcggttcgacagcaacaccccgaatccgaggatggagccacgggcgcggtgggtggagaaggaggggcccgagtattgggatcgcgagacgcgaaactccaaggaaaccgcacagactttccgagcgaacctgaacaccgcactcggctactacaaccagagcgaggccgggtctcacaccgtccaagagatgtacggctgcgacgtggggccggacgggcgtctcctccgcgggttcatgcaggacgcctacgacggcagagattacatcgccctgaacgaggacctgcgctcctggaccgcggcggacacggcggctcagatcaccaagcgcaagtgggaggcggcaggtgatgcggagacttggaggaactacctggagggccggtgcgtggaggggctccgcagatacctggagaacgggaaggacgcgctgctgcgcgcagaccctccaaaggcacatgtgacccatcactccatctctgagcgtgaggtcaccctgaggtgctgggccctgggcttctaccctgaggagatctcactgacctggcagcgtgacggggaggaccagacccaggacatggagcttgtggagaccaggccttcaggggatggaaccttccagaagtgggcagccctggcggtgccttctggagaggagcagagatacacgtgccgtgtgcagcacgaggggcttcaggagcccctcaccctgagatgggaacctcctcagacctccttcctcaccatgggcatcattgttggcctggttctcctcgtagtagctgtggtggctggagctgtgatctggaggaagaagcgctcaggtgaaaaaggacggatctacacccaggctgcaagcagtgacagtacccagggctctgatgtgtctctcacggttcctaaagtttga

SEQ ID NO: 10 - DNA, 1077 bp, Bos taurus
atggggccgcgaaccctcctcctgctgctctcgggggtcctggtcctgaccgagaccctggcgggctcccactccctgaggtatttcctcaccgccgtgtcccggcccggcctcggggaaccccgcttcatcatcgtcggctacgtggacgacacgcagttcgtgcggttcgacagcaacaccccgaatccgaggatggagccacgggcgcggtgggtggagaaggaggggcccgagtattgggatcgcgagacgcgaaactccaaggaaaccgcacagactttccgagcgaacctgaacaacgcactcggctactacaaccagagcgaggccgggtctcacaccgtccaagagatgtacggctgcgacgtggggccggacgggcgtctcctccgcgggttcatgcaggacgcctacgacggcagagattacatcgccctgaacgaggacctgcgctcctggaccgcggcggacacggcggctcagatcaccaagcgcaagtgggaggcggcaggtgatgcggagacttggaggaactacctggagggccggtgcgtggagtggctccgcagatacctggagaacgggaaggacgcgctgctgcgcgcagaccctccaaaggcacatgtgacccatcactccatctctgagcgtgaggtcaccctgaggtgctgggccctgggcttctaccctgaggagatctcactgacctggcagcgtgacggggaggaccagacccaggacatggagcttgtggagaccaggccttcaggggatggaaccttccagaagtgggcagccctggcggtgccttctggagaggagcagagatacacgtgccgtgtgcagcacgaggggcttcaggagcccctcaccctgagatgggaacctcctcagacctccttcctcaccatgggcatcattgttggcctggttctcctcgtagtagctgtggtggctggagctgtgatctggaggaagaagcgctcaggtgaaaaaggacggatctacacccaggctgcaagcagtgacagtacccagggctctgatgtgtctctcacggttcctaaagtttga

SEQ ID NO: 11 - DNA, 1060 bp, Bos taurus
atgctgctgctctcgggggtcctggtcctgaccgagaccgtggcgggctcccactccctgaggtatttcagcaccgccgtgtcccggcccggcctcggggagccccgcttcatcgccgtcggctacgtggacgacacgcagttcacacggttcgacagcgacgccccgaatccaagggaagaaccgcgggcgccgtggatggagcaggaggggctggagtattgggatcgaaacacgagaatctacaaggacacacgcacagcttttccgagtgtacctgaacaccctgcgcggctactacaaccagagcgaggccgggtctcacaccctccagtggatgtccggctgcgacgtggggccgggtgggcgcctcctccgcgggttcatgcagttcggctacgacggcagagattacatcgccctgaaccaggacctgcgctcctggaccgcggcggacacggcggctcagatcaccaagcgcaagtgggaggcggcagataatgcggagagcgagaggaactacctggagggcgagtgcgtggagtggctccgcagatacctggagaacgggaaggacacgctgctgcgcgcagaccctccaaaggcacatgtgacgcatcaccccatctctgaccgtgaggtcaccctgaggtgctgggccctgggcttctaccctgaggagatctcactgacctggcagcgcaatggagaggaccagacccagtacatggagcttgtggagaccaggccttcaggggatggaaccttccagaagtgggcagccctggtggtgcctcctggagaggagcagagatacacgtgccgtgtgcagcacgaggggcttcaggagcccctcaccctgagatgggaacctcctcagacctccttcctcatcatgggcatcattgttggcctggttctcctcgtagtagctctggtggctggagctgtgatctggaggaagaagcgctcaggtgaaaaaggacggatctacacccaggctgcaagcagtgacagtacccagggctctgatgtgtctctcacagttcctaaagctgtc

SEQ ID NO: 12 - DNA, 1077 bp, Bos taurus
atggggccgcgaaccctcctcctgctgctctcgggggtcctggtcctgaccgagaccgtggcgggctcccactccctgaggtatttcagcaccgccgtgtcccggcccggcctcggggagccccgcttcatcaccgtcggctacgtggacgacacgcagttcacacggttcgacagcgacgccccgaatccaagggaagaaccgcgggcgccgtggatggagcaggaggggctggagtattgggatcgaaacacgagaatctacaaggacaccgcacagactttccgagtgtacctgaacaccctgcgcggctactacaaccagagcgaggccgggtctcacaccctccagtggatgtccggctgcgacgtggggccggatgggcgcctcctccgcgggttcatgcagttcggctacgacggcagagattacatcgccctgaaccaggacctgcgctcctggaccgcggcggacacggcggctcagatcaccaagcgcaagtgggaggcggcagataatgcggagagcgagaggaactacctggagggcgagtgcgtggaggggctccgcagatacctggagaacgggaaggacgcgctgctgcgcgcagaccctccaaaggcacatgtgacgcatcaccccatctctgaccgtgaggtcaccctgaggtgctgggccctgggcttctaccctgaggagatctcactgacctggcagcgcaatggagaggaccagacccaggacatggagcttgtggagaccaggccttcaggggatggaaccttccagaagtgggcagccctggtggtgccttctggagaggagcagagatacacgtgccgtgtgcagcagcaggggcttcaggagcccctcaccctgagatgggaacctcctcagacctccttcctcaccatgggcatcattgttggcctggttctcctcgtagtagctgtggtggctggagctgtgatctggaggaagaagcgctcaggtgaaaaaggacggatctacacccaggctgcaagcagtgacagtgcccagggctctgatgtgtctctcacggttcctaaagtttga

SEQ ID NO: 13 - DNA, 1086 bp, Bos taurus
atgcgagttatgaggccgcgaaccctcctcctggtgctctcgcgggtcctggtcctgaccgagaccctggcgggctcccactccctgaggtatttctacaccgccgtgtcccggcccggcctcggggagccccgcttcatcgccgtcggctacgtggacgacacgcagttcacacggttcgacagcgacgccccgaatccaagggacgaaccgcgggtgccgtggatggagcaggaggggccggagtattgggatcgaaacacgagaatctacaaggacaccgcacagattttccgggcgaacctgaacaccgcactcggctactacaaccagagcgaggccgggtctcacaccttccaggagatgtacggctgctacgtggggccggacgggcgcctcctcctcgggttcatgcagttcgcctacgacggcagagattacatcgccctgaacgaggacctgcgctcctggaccgcggcggacacggcggctcagatcaccaagcgcaagtgggaggcggcaggtgaggcggagagacagaggaactacctggagggccggtgcgtggaggggctccgcagatacctggagaacgggaaggacacgctgctgcgcgcagaccctccaaaggcacatgtgacccatcaccccatctctgaccgtgaggtcaccctgaggtgctgggccctgggcttctaccctgaggagatctcactgacctggcagcacgaaggggaggaccagacccaggacatggagcttgtggagaccaggccttcaggggatggaaccttccagaagtgggcagccctggtggtgccttctggagaggagcagagatacacgtgccgtgtgcagcatgaggggcttcaggagcccctcaccctgagatgggaacctcctcagacctccttcctcacaatgggcatcattgttggcctggttctcctcgtagtagctgtggtggctggagctgtgatctggaggaagaagcgctcaggtgaaaaaggacggatctacacccaggctgcaagcagtgacagtgcccagggctctgatgtgtctctcacggttcctaaagtttga

SEQ ID NO: 14 - DNA, 1086 bp, Bos taurus
atgcgagttatgaggccgcgaaccctcctcctgctgctctcgcgggtcctggtcctgaccgagaccctggcgggctcccactccctgaggtatttctacaccgccgtgtcccggcccggcctcggggagccccgcttcatcgccgtcggctacgtggacgacacgcagttcacacggttcgacagcgacgccccgaatccaagggacgaaccgcgggtgcggtggatggagcaggaggggccggagtattgggatcgaaacacgagaatctacaaggacaccgcacagattttccgggcgaacctgaacaccgcactcggctactacaaccagagcgaggccgggtctcacaccttccaggagatgtacggctgctacgtggggccggacgggcgcctcctcctcgggttcatgcagttcgcctacgacggcagagattacatcgccctgaacgaggacctgcgctcctggaccgcggcggacacggcggctcagatcaccaagcgcaagtgggaggcggcaggtgaggcggagagacagaggaactacctggagggccggtgcgtggaggggctccgcagatacctggagaacgggaaggacacgctgctgcgcgcagaccctccaaaggcacatgtgacccatcaccccatctctgaccgtgaggtcaccctgaggtgctgggccctgggcttctaccctgaggagatctcactgacctggcagcacgaaggggaggaccagacccaggacatggagcttgtggagaccaggccttcaggggatggaaccttccagaagtgggcagccctggtggtgccttctggagaggagcagagatacacgtgccgtgtgcagcatgaggggcttcaggagcccctcaccctgagatgggaacctcctcagacctccttcctcacaatgggcatcattgttggcctggttctcctcgtagtagctgtggtggctggagctgtgatctggaggaagaagcgctcaggtgaaaaaggacggatctacacccaggctgcaagcagtgacagtgcccagggctctgatgtgtctctcacggttcctaaagtttga

SEQ ID NO: 15 - DNA, 1086 bp, Bos taurus
atgcgagttatgaggccgcgaaccctcctcctgctgctctcgggggtcctggtcctgaccgagaccctggcgggctcccactccctgaggtatttctacaccggcgtgtcccggcccggcctcggggagccccgcttcatcgccgtcggctacgtggacgacacgcagttcgtgcggttcgacagcgacgccccgaatccaagggaagaaccgcgggtgccgtggatggagcaggaggggccggagtattgggatcgaaacacgagaatctacaaggacaccgcacagattttccgagtggacctgaacaccctgcgcggctactacaaccagagcgagaccgggtctcacaatatccaggcgatgtacggctgcgacgtggggccggacgggcgcctcctccgcgggttctggcagttcggctacgacggcagagattacatcgccctgaacgaggagctgcgctcctggaccgcggcggacacggcggctcagatcaccaagcgcaagtgggaggcggcaggtgctgcggagacttggaggaactacctggagggcgagtgcgtggagtggctccgcagatacctggagaacgggaaggacacgctgctgcgcgcagaccctccaaaggcacatgtgacccatcactccatctctgaccgtgaggtcaccctgaggtgctgggccctgggcttctaccctgaggagatctcactgacctggcagcgcgagggggaggaccagacccaggacatggagcttgtggagaccaggccttcaggggatggaaccttccagaagtgggcagccctggtggtgccttctggagaggagcagagatacacgtgccgtgtgcagcatgaggggcttcaggagcccctaaccctgagatgggaacctcctcagacctccttcctcatcatgggcatcattgttggcctggttctcctcgtagtagctctggtggctggagctgtgatctggaggaagaagcgctcaggtgaaaaaggacggatctacacccaggctgcaagcagtgacagtgcccagggctctgatgtgtctctcacggttcctaaagtgtga

SEQ ID NO: 16 - DNA, 1069 bp, Bos taurus
gcgaaccctcctcctgctactctcgggggtcctggtcctgaccgagacccgggcaggctcccactccctgaggtatttccacaccgccgtgtcccggcccggcctcggggagccgcgcttcatctctgtcggctacgtggacgacacgcagttcgtgcggttcgacagcgactccgcgaatccgagggaagaaccgcgggcgccgtggatggagcaagaggggccggagtattgggatgagcagacgcgaatagtcaaggacaccgcacagtctttccgagtgggcctgaacaccctgcgcggctactacaaccagagcgaggccgggtctcacaccctccagctgatgtacggctgcgacgtggggccggacgggcgccttctccgcgggtatgagcaggacgcctacgacggcagagattacatcgccctgaacgaggacctgcgctcctggaccgcggcggacacggcggctcagatcaccaagcgcaagcgggaggcggcaggtgctgcggagagattaaggaactacctggagggcacgtgcgtggaggggctccgcagatacctggagaacgggaaggacgcgctgctgcgcgcagaccctccaaaggcacatgtgactcatcaccccgtctctgaccgtgaggtcaccctgaggtgctgggccctgggcttctaccctgaggagatctcactgacctggcagcgcgagggggaggaccagacccaggacatggagcttgtggagaccaggccttcaggggatggaaccttccagaagtgggcagccctggtggtgccttctggagaggagcagagatacacgtgccgtgtgcagcatgaggggcttcaggagcccctcaccctgagatgggaacctcctcagacctccttcctcatcatgggcatcattgttggcctggttctcctcgtagtagctctggtggctggagctgtgatctggaggaagaagcgctcaggtgaaaaaggacggatctacacccaggctgcaagcagtgacagtgcccagggctctgatgtgtctctcacggttcctaaagtgtga

SEQ ID NO: 17 - DNA, 1077 bp, Bos taurus
atggggccgggaaccctcttcatgctgctcttgggggccctggtcctgatcgagacccgggctggctcccactccctgaggtatttccacaccgccgtgtctcgacccggcctccgggagcccctctttatcaccgtcggctacgtggacgacacgcagttcgtgcggttcgacagcgacgcccgggatccgaggactgagccacggcagccgtggatggagaaggaggggccggagtattgggatcgcgagactcaaatctccaaggaaaacgcactgtggtaccgagaggccttgaacaacctgcgcggctactacaaccagagcgaggccgggtctcacaccctccaggagatgtacggctgcgacgtggggtcggacgggcgtctccgccgcgggtatgagcagtacggctacgacggcagagattacctcgccctgaacgaggacctgcgctcctggaccgcggcggacacggcggctcagatctccaaacgcaagatggaggcggccggtgctgcggagagattcaggaactacctggagggcacgtgcgtggagtggctccgcagatacctggagaacgggaaggacacgctgctgcgcgcagaccctccaaaggcacatgtgacccgacaccccagctctgagcatgaggtcaccctgaggtgctgggccctgggcttctaccctgaggagatctcactgacctggcagcgcaatggagaggaccagacccaggacatggagcttgtggagaccaggccttcaggggacggaaacttccagaagtgggcagccctggtggtgccttctggagaggagcagagatacacgtgccgtgtgcagcacgaggggcttcaggagcccctcaccctgagatgggaacctcctcagacctccttcctcaccatgggcatcattgttggcctggttctcctcgtagtagctgtggtggctggagctgtgatctggatgaagaagcactcaggtgaaaaaagacggacctacacccaggctgcaagcaatgacagtgcccagggctctgatgtgtctctcacggttcctaaagtgtga

SEQ ID NO: 18 - DNA, 1017 bp, Bos taurus
atggggccgcgaagcctcttcatgctgctcttgggggccctggtcctgatcgagacccgggctggctcccactccctgaggtatttctacaccggcgtgtctcgacccggcctccgggagcccctctttatcaccgtcggctacgtggacgacacgcagttcgtgcggttcgacagcgacgcccgggatccgaggaaagaaccacggcagccgtggatggagaaggaggggccggagtattgggatcgcgagactcaaatctccaaggaaaacgcactgtggtaccgagaggccttgaacaacctgcgcggctactacaaccagagcgaggccgggtctcacaccctccagctgatgtacggctgcgacgtggggccggacgggcgcctcctccgcgggtatacgcagttcggctacgacggcagagattacctcgccctgaacgaggacctgcgctcctggaccgcggcggacacggcggctcagatctccaaacgcaagatggaggcggccggtgatgcggagagacagaggaactacctggagggccggtgcgtggaggggctccgcagatacctggagaacgggaagcatgaggtcaccctgaggtgctgggccctgggcttctaccctgaggagatctcactgacctggcagcgcaatggagaggaccagacccaggacatggagcttgtggagaccaggccttcaggggacggaaacttccagaagtgggcagccctggtggtgccttctggagaggagcagagatacacgtgccgtgtgcagcacgaggggcttcaggagcccctcaccctgagatgggaacctcctcagacctccttcctcaccatgggcatcattgttggcctggttctcctcgtagtagctgtggtggctggagctgtgatctggatgaagaagcactcaggtgaaaaaagacggacctacacccaggctgcaagcaatgacagtgcccagggctctgatgtgtctctcacggttcctaaagtgtga

SEQ ID NO: 19 - DNA, 1077 bp, Bos taurus
atggggccgcgagccctcttcatgctgctcttgggggccctggtcctgatcgagacccgggctggctcccacttcctgaggtatttccacaccgccgtgtctcggcccggcctccgggagcccctctttatcaccgtcggctacgtggacgacacgcagttcgtgcggttcgacagcgacgcccgggatccgaggaaagaaccacggcagccgtggatggagaaggaggggccggagtattgggatcgcgagactcaaatctccaaggaaaacgcactgtggtaccgagaggccttgaacaacctgcgcggctactacaaccagagcgaggccgggtctcacacctatcagcggatgtacggctgcgacgtggggccggacgggcgcctcctcagcgggttcacgcagttcggctacgacggcagagattacatcgccctgaacgaggacctgcgctcctggaccgcggcggacacggcggctcagatcaccaagcgcaagtgggaggcggccggtgaggcggagagattcaggaactacgtggagggccggtgcgtggaggggctccgcagatacctggagaacgggaaggacgcgctgctgcgcgcagaccctccaaaggcacatgtgacccatcaccccatctctgagcatgaggtcaccctgaggtgctgggccctgggcttctaccctgaggagatctcactgacctggcagcgcaatggagaggaccagacccaggacatggagcttgtggagaccaggccttcaggggacggaaacttccagaagtgggcagccctggtggtgccttctggagaggagcagagatacacgtgccgtgtgcagcacgaggggcttcaggagcccctcaccctgagatgggaacctcctcagacctccttcctcaccatgggcatcattgttggcctggttctcctcgtagtagctgtggtggctggagctgtgatctggatgaagaagcactcaggtgaaaaaagacggacctacacccaggctgcaagcaatgacagtgcccagggctctgatgtgtctctcacggttcctaaagtgtga

SEQ ID NO: 20 - DNA, 1086 bp, Bos taurus
atgcgagtcatggggccgcgagccctcttcgtgctgctcttgggggccctggtcctgaccgagacccgggctggctcccactctctgaagtatttccacaccgccgtgtctcggcccggcgacggggagccccgcttcatcaccgttggctacgtggacgacacgcagttcgtgcggttcgacagcgacgccccggatccgaggaaagaaccacgggcgccgtggatagagaaggaggggccggagtattgggatcgcgagacgcgaatctccaaggaaaacacactggtgtatcgagggagcctgaacaacttgcgcggctactacaaccagagcgaggccgggtctcacaccttccagctgatgtacggctgcgacgtggggccggacgggcgcctcctccgcgggttcatgcaggacgcctacgacagcagagattacatcgccctgaacgaggacctgcgctcctggaccgtggcggacacggcggctcagatcaccaagcgcaagtgggaggcggcaggtgttgcggagagattcaggaactacgtcgagggccggtgcgtggaggggctccgcagatacctggagaacgggaaggacgcgctgctgcgcgcagaccctccaaaggcacatgtgacccatcaccccatctctgagcgtgaggtcaccctgaggtgctgggccctgggcttctaccctaaggagatctcactgacctggcagcgcaatggagaggaccagacccaggacatggagcttgtggagaccaggccttcaggggacggaaacttccagaagtgggcggccctggtggtgccttctggagaggaacagagatacacgtgccatgtgcagcacgaagggcttcaggagcccctcaccctgagatgggaacctcctcagacctccttcctcatcatgggcatcattgttggcctggttctcctcgtagtagctgtggtggctggagctgtgatctggaggaagaagcgctcaggtgaaaaaagacagacctacacccaggctgcaagtggtgacagtgaccagggctctgatgtgtctctcacggttcctaaagtttga

SEQ ID NO: 21 - DNA, 1086 bp, Bos taurus
atgcgagtcatggggccgcgaaccctcttcgtgctgctcttgggggccctggtcctgaccgagacccgggctggctcccactctctgaagtatttccacaccgccgtgtctcggcccggcgacggggagccccgcttcatcaccgttggctacgtggacgacacgcagttcgtgcggttcgacagcgacgccccggatccgaggaaagaaccacgggcgccgtggatagagaaggaggggccggagtattgggatcgcgagacgcgaatctccaaggaaaacacactggtgtatcgagggagcctgaacaacttgcgcggctactacaaccagagcgaggccgggtctcacacctatcaggacatgtacggctgcgacgtggggccggacgggcgcctcctcggcgggtatgatcagtacggctacgacggcagagattacctcgccctgaacgaggacctgcgctcctggaccgcggcggacacggcggctcagatcaccaagcgcaagtgggaggcggcaggtgttgcggagagattcaggaactacgtcgagggccggtgcgtggaggggctccgcagatacctggagaacgggaaggacgcgctgctgcgcgcagaccctccaaaggcacatgtgacccatcaccccatctctgagcgtgaggtcaccctgaggtgctgggccctgggcttctaccctaaggagatctcactgacctggcagcgcaatggagaggaccagacccaggacatggagcttgtggagaccaggccttcaggggacggaaacttccagaagtgggcggccctggtggtgccttctggagaggaacagagatacacgtgccatgtgcagcacgaagggcttcaggagcccctcaccctgagatgggaacctcctcagacctccttcctcatcatgggcatcattgttggcctggttctcctcgtagtagctgtggtggctggagctgtgatctggaggaagaagcgctcaggtgaaaaaagacagacctacacccaggctgcaagtggtgacagtgaccagggctctgatgtgtctctcacggttcctaaagtttga

SEQ ID NO: 22 - DNA, 1086 bp, Bos taurus
atgcgagtcatggggccgcgaaccctcttcgtgctgctcttgggggccctggtcctgaccgagacccgggctggctcccactccctgaggtatttctacaccgccgtgtcccggcccggcgacggggagccccgcttcatcaccgttggctacgtggacgacacgcagttcgtgtggttcgacagcgacgccccggatccgaggaaagaaccacggacgccgtggatagagaaggaggggccggagtattgggatcgcgagacgcgaatctccaaggaaaacacactggtgtatcgagggagcctgaacaacttgcgcggctactacaaccagagcgaggccgggtctcacaccttccagcagatgttcggctgcgacgtggggccggacgggcgcctcctcggcgggtacaggcagtacgcctacgacggcagagattacatcgccctgaacgaggacctgcgctcctggaccgcggcggacacggcggctcagatcaccaagcgcaagtgggaggcggcaggtgatgcggagagattcaggaactacgtggagggcgagtgcgtggaggggctccgcagatacctggagaacgggaaggacacactgctgcgcgcagaccctccaaaggcacatgtgacccatcaccccatctctgagcgtgaggtcaccctgaggtgctgggccctgggcttctaccctgaggagatctcactgacctggcagcgcaatggagaggaccagacccaggacatggagcttgtggagaccaggccttcaggggacggaaacttccagaagtgggcggccctggtggtgccttctggagaggaacagagatacacgtgccatgtgcagcacaaagggcttcaggagcccctcaccctgagatgggaacctcctcagacctccttcctcaccatgggcatcattgttggcctggttctcctcgtagtagctgtggtggctggagctgtgatctggaggaagaagcgctcaggtgaaaaaagacagacctacacccaggctgcaagtggtgacagtgaccagggctctgatgtgtctctcacggttcctaaagtttga

SEQ ID NO: 23 - DNA, 942 bp, Bos taurus
gttggctacgtggacgacacgcagttcgtgcggttcgacagcgacgccccggatccgaggaaagaaccacgggcgccgtggatagagaaggaggggccggagtattgggatcgcgagacgcgaatctccaaggaaaacacactggtgtatcgagggagcctgaacaacttgcgcggctactacaaccagagcgaggccgggtctcacaccttccagcagatgtacggctgcgacgtggggccggacggacgcctcctccgcgggttcaagcagttcgcctacgacagcagagattacatcgccctgaacgaggagctgcgctcctggaccgcggcggacacggcggctcagatcaccaagcgcaagtgggaggcggcaggtgctgcggagacttggaggaactacctggagggcgagtgcgtggagtggctccgcagatacctggagaacgggaaggacacgctgctgcgcgcagaccctccaaaggcacatgtgacccatcaccccatctctgaccgtgaggtcaccctgaggtgctgggccctgggcttctaccctaaggagatctcactgacctggcagcgcaatggagaggaccagacccaggacatggagcttgtggagaccaggccttcaggggacggaaacttccagaagtgggcggccctggtggtgccttctggagaggaacagagatacacgtgccatgtgcagcacgaagggcttcaggagcccctcaccctgagatgggaacctcctcagacctccttcctcatcatgggcatcattgttggcctggttctcctcgtagtagctgtggtggctggagctgtgatctggaggaagaagcgctcaggtgaaaaaagacagacctacacccaggctgcaagtggtgacagtgaccagggctctgatgtgtctctcacggttcctaaagtgtga

SEQ ID NO: 24 - DNA, 1083 bp, Bos taurus
cgagtcatggggccgcgaaccctcttcgtgctgctcttgggggccctggccctgaccgagacccgggctggctcccactccctgaggtatttctacaccgccgtgtctcggcccggcctcggggagccccgcttcatctccgtcggctacgtggacgacacgcagttcgtgcggttcgacagcgacgccccgaatccaagggaagaaccgcgggcgccgtggatagagaaggaagggccggagtattgggatcgcgagacgcgaatctccaaggaaaacacactggtgtatcgagagagcctgaacaacctgcgcggctactacaaccagagcgaggccgggtctcacaatatccaggcgatgtacggctgcgacgtggggtcggacgggagtttcctccgcgggtacagtcaggacgcctacgacggcagagattacatcgccctgaacgaggacctgcgctcctggaccgcggcggacacggcggctcagatcaccaagcgcaagtgggaggcggaaggttatgctgagtctttgaggaactacctggagggccggtgcgtggagtggctccgcagatacctggagaacgggaaggacgcgctgctgcgcgcagaccctccaatggcacatgtgacccatcaccccagctctgagcgtgaggtcaccctgaggtgctgggccctgggcttctaccctaaggagatctcactgacctggcagcgcgagggggaggaccagacccaggacatggagcttgtggagaccaggccttcaggggatggaaccttccagaagtgggcagccctggtggtgccttctggagaggagcagagatacacgtgccatgtgcagcacgaagggcttcaggagcccctcatcctgagatgggaacctcctcagacctccttcctcatcatgggcatcattgttggcctggttctcctcgtagtagctgtggtggctggagctgtgatctggaggaagaagcgctcaggtgaaaaaagacagacctacacccaggctgcaagtggtgacagtgaccagggctctgatgtgtctctcacggttcctaaagtgtga

SEQ ID NO: 25 - DNA, 1014 bp, Bos taurus
ggctcccactcgctgaggtatttcctcacccgcgtgtcccggcccgacctcggggagccccggtacctggaagtcggctacgtggacaacacgcagttcgtgcggttcgacagcgacgcccggaatccgaggatggagccgcgggcgcggtgggtggagcaggaggggccggagtattgggatcagaacacgcgaaactccaagggcgccgcacagactttccgagtggacctgaacaccctgcgcggctactacaaccagagcgaggccgggtctcacactatccaggcgatgtacggctgcgacgtggggccggacgggcgtttcctccgcgggtacaggcaggacgcttacgacggcagagattacatcgccctgaacgaggacctgcgctcctggaccgcggcggacacggcggctcagatcaccaagcgcaagtgggaggaggcaggtgctgcggagggcgagaggaactacctggagggcacgtgcgtggagtggctccgcagatacctggagaacgggaaggacgcgctgctgcgcgcagaccctccaaaggcacatgtgacccatcaccccatctctgaccgtgaggtcaccctgaggtgctgggccctgggcttctaccctgaggagatctcactgacctggcagcgcgagggggaggaccagacccaggacatggagcttgtggagaccaggccttcaggggacggaagcttccagaagtgggcagccctggtggtgccttctggagaggagcagagatacacgtgccgtgtgcagcacaaagggcttcaggagcccctcaccctgatatgggaacctcctcagccctccgttcccatcatgggcatcgttgttggcctggttctcctcctggtggctgtagtggctggagctgtgatctggaggaagaagtgctcaggtgaaaaaggacagacctacactcaggctgcaagcagtgacagtgaccagggctctgatgtgtctcgcacggttcctaaagtgtga

SEQ ID NO: 26 - DNA, 1061 bp, Bos taurus
tccagctgctctcgggggccctggtcctgaccgagaccttggctggctcccactccctgaggtatttcctcaccgccgtgtcccggcccggcgtcagggagccccgcttcatcatcgtcggctacgtggacgacttgcagttcgtgcggttcgacagcgacgccggggatccgagagtggagccgcgggcgcggtgggtggagaaagaggggcccgagtattgggatgaggagacgtggagagccaaggaccacgcacaatttttccgacggggcctgaacgccctgcgcggctactataaccagagcgaggccgggtctcacactttccaggagatgtacggctgctacgtggggccggacgggcgcctcctccgcggctatgagcagttcggctacgaaggcagagattacatcgccctgaacggagacctgcactcctggaccgcggcggacacggcggctcagatctccaaacgcaagtgggaggcagcgggttctacggacttttacaggaactaccttgagggcgagtgcgtggagtggctccgcagatacctggagaacgggaaggacacgttgctgcgcgcagaccctccaaaggcacatgtgacccatcaccccatctctgagcgtgaggtcaccctgaggtgctgggccctgggcttctaccctgaggagatctcactgacctggcagcatgatggggaggaccagacccaggacatggagcttgtggagaccaggccttcaggggatggaaccttccagaagtgggtggccctggtggtgccttctggagaagagcagagatacacgtgccgtgtgcagcacgaggggcttcaggagcccctcaccctgagatgggaacctcctcagacctccttcctcaccatgggcatcattgttggtctggttctccttgtcactggagctgtggtggctggatttgtgatctggatgaagaagcgctcaggtgaaaaaggagggaattatatccaggcttcaagcagtgccagtgcccggggctctgatgtgtctctcacggttcctaaagtgtga

SEQ ID NO: 27 - DNA, 1089 bp, Bos taurus
atgcgggtcgtggggccgagaaccctcctcctgctgctgccgggtgcgctgatcctcactgagaccagggcggggtcccactccctgaggtatttctacaccgccgtgtcccggcccggcctcggggagccccgcttcatctccgtcggctacgtggacgacacgcagttcgtgcggttcgacagcgacgccccggatccgaggatagagcctacagcgcggtgggtggagcaggaggggcccgagtactggcatcaggagacgcagagaactaaggacaccgcacaatttttccgagtgtacctgaacaccctgcgcggctactacaaccagagcgaggccgggtctcacacagtccaagagatgtacggctgcgacgtggggccggacgggcttctccgcgggtatgatcagttcgcctacgacggcagagattacatcgccctgaacgaggacctgcgttcctggaccgcggcggacacggcggctcaagtcaccaagcacaatgctgaggcggcaggtgatgccgcgcgtgtgaggatctacctggagggcaagtgtgtggagtggctccgcagatacctggtgaccgggaaggacacgctgctgcgcgcagaccctccaaagacacatgtggcccatcaccccatctctgaccatgaggtcaccttgaggtgctgggccctgggcttctatcccgatgagatctcactgacctggcagcgtgacggggaggaccagactcaggacatggagcttgtggagaccaggccttcaggggatggaaccttccagaagtgggcggccctggtggtgccttctggagaggagcagagatacacgtgccatgtgcagcacgaggggcttcaggagcccctcaccctgagatgggaacctcctcagccctccatccccatcatgggcatcattgttggcctggttctcctcatggtcactggagctgtggtgactggagctatgatttggaggaagaagcactcaggtgaaaaaggaaggggctacacccagactgcaagcagttacagtgaccagggctctgatgtgtctctcatggttcctaaagtgtga

SEQ ID NO: 28 - DNA, 20 bp, Artificial Sequence
  Description of Artificial Sequence  primer
acgtggacgacacgcagttc

SEQ ID NO: 29 - DNA, 20 bp, Artificial Sequence
  Description of Artificial Sequence  primer
acgtggacgacacggagttc

SEQ ID NO: 30 - DNA, 21 bp, Artificial Sequence
  Description of Artificial Sequence  primer
ctcgctctggttgtagtagcc

SEQ ID NO: 31 - DNA, 25 bp, Artificial Sequence
  Description of Artificial Sequence  primer
tggtcggggcgggtcagggtctcac

SEQ ID NO: 32 - DNA, 29 bp, Artificial Sequence
  Description of Artificial Sequence  primer
ccttcccgttctccaggtatctgcggagc

SEQ ID NO: 33 - DNA, 18 bp, Artificial Sequence
  Description of Artificial Sequence BoLA Class I, Exon 2, Series A Probe
cgggagacgcaaagggcc

SEQ ID NO: 34 - DNA, 18 bp, Artificial Sequence
  Description of Artificial Sequence BoLA Class I, Exon 2, Series A Probe
caggagacgcgaaaggcc

SEQ ID NO: 35 - DNA, 18 bp, Artificial Sequence
  Description of Artificial Sequence BoLA Class I, Exon 2, Series A Probe
cggaacacgcgaaacgcc

SEQ ID NO: 36 - DNA, 22 bp, Artificial Sequence
  Description of Artificial Sequence BoLA Class I, Exon 2, Series A Probe
atcgaaacacgagaatctacaa

SEQ ID NO: 37 - DNA, 18 bp, Artificial Sequence
  Description of Artificial Sequence BoLA Class I, Exon 2, Series A Probe
gagcagacgcgaatagtc

SEQ ID NO: 38 - DNA, 18 bp, Artificial Sequence
  Description of Artificial Sequence BoLA Class I, Exon 2, Series A Probe
cgcgagacgcgaaactcc

SEQ ID NO: 39 - DNA, 18 bp, Artificial Sequence
  Description of Artificial Sequence BoLA Class I, Exon 2, Series A Probe
cgcgagactcaaatctcc

SEQ ID NO: 40 - DNA, 18 bp, Artificial Sequence
  Description of Artificial Sequence BoLA Class I, Exon 2, Series A Probe
cgcgagacgcgaatctcc

SEQ ID NO: 41 - DNA, 18 bp, Artificial Sequence
  Description of Artificial Sequence BoLA Class I, Exon 2, Series A Probe
cagaacacgcgaaactcc

SEQ ID NO: 42 - DNA, 18 bp, Artificial Sequence
  Description of Artificial Sequence BoLA Class I, Exon 2, Series A Probe
gaggagacgtggagagcc

SEQ ID NO: 43 - DNA, 18 bp, Artificial Sequence
  Description of Artificial Sequence BoLA Class I, Exon 2, Series A Probe
caggagacgcagagaact

SEQ ID NO: 44 - DNA, 18 bp, Artificial Sequence
  Description of Artificial Sequence BoLA Class I, Exon 2, Series A Probe
caagagacgcggatacaa

SEQ ID NO: 45 - DNA, 18 bp, Artificial Sequence
  Description of Artificial Sequence BoLA Class I, Exon 2, Series A Probe
caggcgacgcagagaact

SEQ ID NO: 46 - DNA, 18 bp, Artificial Sequence
  Description of Artificial Sequence BoLA Class I, Exon 2, Series A Probe
caggagacgcgaaacgcc

SEQ ID NO: 47 - DNA, 18 bp, Artificial Sequence
  Description of Artificial Sequence BoLA Class I, Exon 2, Series A Probe
gagatgacacgagatgcc

SEQ ID NO: 48 - DNA, 18 bp, Artificial Sequence
  Description of Artificial Sequence BoLA Class I, Exon 2, Series A Probe
gacgagacgcgaatctcc

SEQ ID NO: 49 - DNA, 20 bp, Artificial Sequence
  Description of Artificial Sequence BoLA Class I, Exon 2, Series A Probe
cggtgtccttgaagtttcgc

SEQ ID NO: 50 - DNA, 19 bp, Artificial Sequence
  Description of Artificial Sequence BoLA Class I, Exon 2, Series A Probe
cggcgccctttaagtttcg

SEQ ID NO: 51 - DNA, 18 bp, Artificial Sequence
  Description of Artificial Sequence BoLA Class I, Exon 2, Series A Probe
gcgatgacaagagatgcc

SEQ ID NO: 52 - DNA, 18 bp, Artificial Sequence
  Description of Artificial Sequence BoLA Class I, Exon 2, Series A Probe
cagaacacgcgaaacgcc

SEQ ID NO: 53 - DNA, 18 bp, Artificial Sequence
  Description of Artificial Sequence BoLA Class I, Exon 2, Series A Probe
caggagacgcagaggact

SEQ ID NO: 54 - DNA, 21 bp, Artificial Sequence
  Description of Artificial Sequence BoLA Class I, Exon 2, Series A Probe
catcaggagacgcagataact

SEQ ID NO: 55 - DNA, 18 bp, Artificial Sequence
  Description of Artificial Sequence BoLA Class I, Exon 2, Series A Probe
gaggaaacgcaaagggcc

SEQ ID NO: 56 - DNA, 18 bp, Artificial Sequence
  Description of Artificial Sequence BoLA Class I, Exon 2, Series A Probe
gaggagacgcaaagggcc

SEQ ID NO: 57 - DNA, 20 bp, Artificial Sequence
  Description of Artificial Sequence BoLA Class I, Exon 2, Series A Probe
tcgaaacacgaggatctaca

SEQ ID NO: 58 - DNA, 19 bp, Artificial Sequence
  Description of Artificial Sequence BoLA Class I, Exon 2, Series B Probe
cagattttccgagtgagcc

SEQ ID NO: 59 - DNA, 20 bp, Artificial Sequence
  Description of Artificial Sequence BoLA Class I, Exon 2, Series B Probe
caatttttccgagtgagcct

SEQ ID NO: 60 - DNA, 18 bp, Artificial Sequence
  Description of Artificial Sequence BoLA Class I, Exon 2, Series B Probe
cagactttccgggcgaac

SEQ ID NO: 61 - DNA, 20 bp, Artificial Sequence
  Description of Artificial Sequence BoLA Class I, Exon 2, Series B Probe
cagagtttccgagtgaacct

SEQ ID NO: 62 - DNA, 19 bp, Artificial Sequence
  Description of Artificial Sequence BoLA Class I, Exon 2, Series B Probe
cagactttccgagtggacc

SEQ ID NO: 63 - DNA, 18 bp, Artificial Sequence
  Description of Artificial Sequence BoLA Class I, Exon 2, Series B Probe
cagactttccgagcgaac

SEQ ID NO: 64 - DNA, 19 bp, Artificial Sequence
  Description of Artificial Sequence BoLA Class I, Exon 2, Series B Probe
cagactttccgagtgtacc

SEQ ID NO: 65 - DNA, 18 bp, Artificial Sequence
  Description of Artificial Sequence BoLA Class I, Exon 2, Series B Probe
cagattttccgggcgaac

SEQ ID NO: 66 - DNA, 19 bp, Artificial Sequence
  Description of Artificial Sequence BoLA Class I, Exon 2, Series B Probe
cagattttccgagtggacc

SEQ ID NO: 67 - DNA, 18 bp, Artificial Sequence
  Description of Artificial Sequence BoLA Class I, Exon 2, Series B Probe
cagtctttccgagtgggc

SEQ ID NO: 68 - DNA, 18 bp, Artificial Sequence
  Description of Artificial Sequence BoLA Class I, Exon 2, Series B Probe
ctgtggtaccgagaggcc

SEQ ID NO: 69 - DNA, 18 bp, Artificial Sequence
  Description of Artificial Sequence BoLA Class I, Exon 2, Series B Probe
ctggtgtatcgagggagc

SEQ ID NO: 70 - DNA, 19 bp, Artificial Sequence
  Description of Artificial Sequence BoLA Class I, Exon 2, Series B Probe
actggtgtatcgagagagc

SEQ ID NO: 71 - DNA, 19 bp, Artificial Sequence
  Description of Artificial Sequence BoLA Class I, Exon 2, Series B Probe
ctggtatatcgagagagcc

SEQ ID NO: 72 - DNA, 19 bp, Artificial Sequence
  Description of Artificial Sequence BoLA Class I, Exon 2, Series B Probe
caatttttccgacggggcc

SEQ ID NO: 73 - DNA, 21 bp, Artificial Sequence
  Description of Artificial Sequence BoLA Class I, Exon 2, Series B Probe
acaatttttccgagtgtacct

SEQ ID NO: 74 - DNA, 19 bp, Artificial Sequence
  Description of Artificial Sequence BoLA Class I, Exon 2, Series B Probe
cagaatttccgagtgggcc

SEQ ID NO: 75 - DNA, 18 bp, Artificial Sequence
  Description of Artificial Sequence BoLA Class I, Exon 2, Series B Probe
cagactttccgagcaaac

SEQ ID NO: 76 - DNA, 19 bp, Artificial Sequence
  Description of Artificial Sequence BoLA Class I, Exon 2, Series B Probe
ctgctgtatcgagagaacc

SEQ ID NO: 77 - DNA, 18 bp, Artificial Sequence
  Description of Artificial Sequence BoLA Class I, Exon 2, Series B Probe
ctgaagtaccgagaggcc

SEQ ID NO: 78 - DNA, 21 bp, Artificial Sequence
  Description of Artificial Sequence BoLA Class I, Exon 2, Series B Probe
cagaaatcccgattatgcttg

SEQ ID NO: 79 - DNA, 20 bp, Artificial Sequence
  Description of Artificial Sequence BoLA Class I, Exon 2, Series B Probe
caggaatcccgattatgctt

SEQ ID NO: 80 - DNA, 20 bp, Artificial Sequence
  Description of Artificial Sequence BoLA Class I, Exon 2, Series B Probe
ctgctgtatcgaaagaacct

SEQ ID NO: 81 - DNA, 18 bp, Artificial Sequence
  Description of Artificial Sequence BoLA Class I, Exon 2, Series B Probe
cagagattgcgaacgggc

SEQ ID NO: 82 - DNA, 19 bp, Artificial Sequence
  Description of Artificial Sequence BoLA Class I, Exon 2, Series B Probe
cagactttccgagtgaacc

SEQ ID NO: 83 - DNA, 21 bp, Artificial Sequence
  Description of Artificial Sequence BoLA Class I, Exon 2, Series B Probe
cagagatcccaattatgcttg

SEQ ID NO: 84 - DNA, 19 bp, Artificial Sequence
  Description of Artificial Sequence BoLA Class I, Exon 2, Series B Probe
cagtctttccgagtgaacc

SEQ ID NO: 85 - DNA, 20 bp, Artificial Sequence
  Description of Artificial Sequence BoLA Class I, Exon 2, Series B Probe
cagtttccgagtgaacctga

SEQ ID NO: 86 - DNA, 20 bp, Artificial Sequence
  Description of Artificial Sequence BoLA Class I, Exon 2, Series B Probe
caggttttccaagtgaacct

SEQ ID NO: 87 - DNA, 19 bp, Artificial Sequence
  Description of Artificial Sequence BoLA Class I, Exon 2, Series B Probe
caggttttccgagtgaacc

SEQ ID NO: 88 - DNA, 18 bp, Artificial Sequence
  Description of Artificial Sequence BoLA Class I, Exon 3, Series A Probe
ggcgtcctgcctgtatcc

SEQ ID NO: 89 - DNA, 18 bp, Artificial Sequence
  Description of Artificial Sequence BoLA Class I, Exon 3, Series A Probe
gccgaactgctcatagcc

SEQ ID NO: 90 - DNA, 18 bp, Artificial Sequence
  Description of Artificial Sequence BoLA Class I, Exon 3, Series A Probe
ggcgttctgccagatccc

SEQ ID NO: 91 - DNA, 17 bp, Artificial Sequence
  Description of Artificial Sequence BoLA Class I, Exon 3, Series A Probe
ggcgtcctgcctgtacc

SEQ ID NO: 92 - DNA, 18 bp, Artificial Sequence
  Description of Artificial Sequence BoLA Class I, Exon 3, Series A Probe
agcgtcctgcctgtaccc

SEQ ID NO: 93 - DNA, 18 bp, Artificial Sequence
  Description of Artificial Sequence BoLA Class I, Exon 3, Series A Probe
ggcgtactgcctgtaccc

SEQ ID NO: 94 - DNA, 18 bp, Artificial Sequence
  Description of Artificial Sequence BoLA Class I, Exon 3, Series A Probe
ggcgtcctgactgtaccc

SEQ ID NO: 95 - DNA, 18 bp, Artificial Sequence
  Description of Artificial Sequence BoLA Class I, Exon 3, Series A Probe
ggcgaactgatcgtaccc

SEQ ID NO: 96 - DNA, 19 bp, Artificial Sequence
  Description of Artificial Sequence BoLA Class I, Exon 3, Series A Probe
ggcgagctgattatacccg

SEQ ID NO: 97 - DNA, 19 bp, Artificial Sequence
  Description of Artificial Sequence BoLA Class I, Exon 3, Series A Probe
ggcgtcctgattatacccg

SEQ ID NO: 98 - DNA, 18 bp, Artificial Sequence
  Description of Artificial Sequence BoLA Class I, Exon 3, Series A Probe
gccgaactgcgtataccc

SEQ ID NO: 99 - DNA, 18 bp, Artificial Sequence
  Description of Artificial Sequence BoLA Class I, Exon 3, Series A Probe
ggcgtcctgctcataccc

SEQ ID NO: 100 - DNA, 18 bp, Artificial Sequence
  Description of Artificial Sequence BoLA Class I, Exon 3, Series A Probe
gccgtactgctcataccc

SEQ ID NO: 101 - DNA, 19 bp, Artificial Sequence
  Description of Artificial Sequence BoLA Class I, Exon 3, Series A Probe
gccgtactgatcatacccg

SEQ ID NO: 102 - DNA, 19 bp, Artificial Sequence
  Description of Artificial Sequence BoLA Class I, Exon 3, Series A Probe
ggctaactgatcatacccg

SEQ ID NO: 103 - DNA, 19 bp, Artificial Sequence
  Description of Artificial Sequence BoLA Class I, Exon 3, Series A Probe
ggcgaactgatcatacccg

SEQ ID NO: 104 - DNA, 19 bp, Artificial Sequence
  Description of Artificial Sequence BoLA Class I, Exon 3, Series A Probe
gccgtactgctaatacccg

SEQ ID NO: 105 - DNA, 18 bp, Artificial Sequence
  Description of Artificial Sequence BoLA Class I, Exon 3, Series A Probe
ggcgaactgcttgaaccc

SEQ ID NO: 106 - DNA, 18 bp, Artificial Sequence
  Description of Artificial Sequence BoLA Class I, Exon 3, Series A Probe
gccgaactgcgtgaaccc

SEQ ID NO: 107 - DNA, 18 bp, Artificial Sequence
  Description of Artificial Sequence BoLA Class I, Exon 3, Series A Probe
ggcgtcctgcatgaaccc

SEQ ID NO: 108 - DNA, 18 bp, Artificial Sequence
  Description of Artificial Sequence BoLA Class I, Exon 3, Series A Probe
gccgtactgcatgaaccc

SEQ ID NO: 109 - DNA, 18 bp, Artificial Sequence
  Description of Artificial Sequence BoLA Class I, Exon 3, Series A Probe
ggcgaactgcatgaaccc

SEQ ID NO: 110 - DNA, 18 bp, Artificial Sequence
  Description of Artificial Sequence BoLA Class I, Exon 3, Series A Probe
gccgaactgcatgaaccc

SEQ ID NO: 111 - DNA, 18 bp, Artificial Sequence
  Description of Artificial Sequence BoLA Class I, Exon 3, Series A Probe
gccgaactgccagaaccc

SEQ ID NO: 112 - DNA, 18 bp, Artificial Sequence
  Description of Artificial Sequence BoLA Class I, Exon 3, Series A Probe
gccgaactgccaaaaccc

SEQ ID NO: 113 - DNA, 19 bp, Artificial Sequence
  Description of Artificial Sequence BoLA Class I, Exon 3, Series A Probe
ggcgaactgatcataccgc

SEQ ID NO: 114 - DNA, 19 bp, Artificial Sequence
  Description of Artificial Sequence BoLA Class I, Exon 3, Series A Probe
ggccttctgccagaatcca

SEQ ID NO: 115 - DNA, 18 bp, Artificial Sequence
  Description of Artificial Sequence BoLA Class I, Exon 3, Series B Probe
cgctgaggagagacacac

SEQ ID NO: 116 - DNA, 19 bp, Artificial Sequence
  Description of Artificial Sequence BoLA Class I, Exon 3, Series B Probe
ggcaggcaaagatccaacg

SEQ ID NO: 117 - DNA, 18 bp, Artificial Sequence
  Description of Artificial Sequence BoLA Class I, Exon 3, Series B Probe
ggaggcagagttccaacg

SEQ ID NO: 118 - DNA, 18 bp, Artificial Sequence
  Description of Artificial Sequence BoLA Class I, Exon 3, Series B Probe
taatgcggagagcgagag

SEQ ID NO: 119 - DNA, 18 bp, Artificial Sequence
  Description of Artificial Sequence BoLA Class I, Exon 3, Series B Probe
taatgcggagagcgggag

SEQ ID NO: 120 - DNA, 18 bp, Artificial Sequence
  Description of Artificial Sequence BoLA Class I, Exon 3, Series B Probe
tcgtgcggagagattcag

SEQ ID NO: 121 - DNA, 19 bp, Artificial Sequence
  Description of Artificial Sequence BoLA Class I, Exon 3, Series B Probe
gtgaagctgaggtacagag

SEQ ID NO: 122 - DNA, 18 bp, Artificial Sequence
  Description of Artificial Sequence BoLA Class I, Exon 3, Series B Probe
tgaggcggagagacacag

SEQ ID NO: 123 - DNA, 18 bp, Artificial Sequence
  Description of Artificial Sequence BoLA Class I, Exon 3, Series B Probe
tgaggcggagagacgcag

SEQ ID NO: 124 - DNA, 18 bp, Artificial Sequence
  Description of Artificial Sequence BoLA Class I, Exon 3, Series B Probe
tgaggcggagagattcag

SEQ ID NO: 125 - DNA, 18 bp, Artificial Sequence
  Description of Artificial Sequence BoLA Class I, Exon 3, Series B Probe
tgatgccgcgcgtgtgag

SEQ ID NO: 126 - DNA, 19 bp, Artificial Sequence
  Description of Artificial Sequence BoLA Class I, Exon 3, Series B Probe
gtgatgcggagacttggag

SEQ ID NO: 127 - DNA, 19 bp, Artificial Sequence
  Description of Artificial Sequence BoLA Class I, Exon 3, Series B Probe
gtgatgcggagagacagag

SEQ ID NO: 128 - DNA, 20 bp, Artificial Sequence
  Description of Artificial Sequence BoLA Class I, Exon 3, Series B Probe
ggtgatgcggagagattaag

SEQ ID NO: 129 - DNA, 19 bp, Artificial Sequence
  Description of Artificial Sequence BoLA Class I, Exon 3, Series B Probe
gtgatgcggagagattcag

SEQ ID NO: 130 - DNA, 18 bp, Artificial Sequence
  Description of Artificial Sequence BoLA Class I, Exon 3, Series B Probe
tgatgcggagggacacag

SEQ ID NO: 131 - DNA, 18 bp, Artificial Sequence
  Description of Artificial Sequence BoLA Class I, Exon 3, Series B Probe
tgatgcggcgcgtgtgag

SEQ ID NO: 132 - DNA, 18 bp, Artificial Sequence
  Description of Artificial Sequence BoLA Class I, Exon 3, Series B Probe
tgctgcgaagggcgagag

SEQ ID NO: 133 - DNA, 18 bp, Artificial Sequence
  Description of Artificial Sequence BoLA Class I, Exon 3, Series B Probe
tgctgcggagacttggag

SEQ ID NO: 134 - DNA, 18 bp, Artificial Sequence
  Description of Artificial Sequence BoLA Class I, Exon 3, Series B Probe
tgctgcggagagacagag

SEQ ID NO: 135 - DNA, 19 bp, Artificial Sequence
  Description of Artificial Sequence BoLA Class I, Exon 3, Series B Probe
gtgctgcggagagattaag

SEQ ID NO: 136 - DNA, 18 bp, Artificial Sequence
  Description of Artificial Sequence BoLA Class I, Exon 3, Series B Probe
tgctgcggagagattcag

SEQ ID NO: 137 - DNA, 18 bp, Artificial Sequence
  Description of Artificial Sequence BoLA Class I, Exon 3, Series B Probe
tgctgcggagcgtgtgag

SEQ ID NO: 138 - DNA, 17 bp, Artificial Sequence
  Description of Artificial Sequence BoLA Class I, Exon 3, Series B Probe
tgctgcggagggcgaga

SEQ ID NO: 139 - DNA, 19 bp, Artificial Sequence
  Description of Artificial Sequence BoLA Class I, Exon 3, Series B Probe
gtgttgcggagagattcag

SEQ ID NO: 140 - DNA, 19 bp, Artificial Sequence
  Description of Artificial Sequence BoLA Class I, Exon 3, Series B Probe
gttacgctgaggtacagag

SEQ ID NO: 141 - DNA, 19 bp, Artificial Sequence
  Description of Artificial Sequence BoLA Class I, Exon 3, Series B Probe
gttatgctgaggtacagag

SEQ ID NO: 142 - DNA, 21 bp, Artificial Sequence
  Description of Artificial Sequence BoLA Class I, Exon 3, Series B Probe
cagattatgctgagtctttga

SEQ ID NO: 143 - DNA, 20 bp, Artificial Sequence
  Description of Artificial Sequence BoLA Class I, Exon 3, Series B Probe
ggttctacggacttttacag

SEQ ID NO: 144 - DNA, 18 bp, Artificial Sequence
  Description of Artificial Sequence BoLA Class I, Exon 3, Series B Probe
ttctgcggagagcgggag

SEQ ID NO: 145 - DNA, 21 bp, Artificial Sequence
  Description of Artificial Sequence BoLA Class I, Exon 3, Series B Probe
aaggttatgctgagtctttga